(19) United States
(12) Patent Application Publication
(10) Pub. No.: US 2023/0242912 A1
(43) Pub. Date: Aug. 3, 2023

(54) METHODS AND COMPOSITIONS RELATED TO CATALYTIC ACTIVATION OF HUMAN ARGONAUTE-3

(71) Applicant: OHIO STATE INNOVATION FOUNDATION, Columbus, OH (US)

(72) Inventors: Kotaro NAKANISHI, Columbus, OH (US); Mi Seul PARK, New York, NY (US)

(21) Appl. No.: 18/011,799

(22) PCT Filed: Jun. 30, 2021
(86) PCT No.: PCT/US2021/039897

§ 371 Date: Dec. 20, 2022

Related U.S. Application Data
(60) Provisional application No. 63/110,405, filed on Nov. 6, 2020.
(60) Provisional application No. 63/046,024, filed on Jun. 30, 2020.

(51) Int. Cl. C12N 15/113; A61K 48/00

(52) U.S. Cl. CPC C12N 15/113; A61K 48/0066; C12N 2330/30; C12N 2310/141

(57) ABSTRACT

Disclosed herein are methods and compositions related to the use of the Argonaute-3 (AGO3) polypeptide in disease detection, prevention, and treatment. Specifically, disclosed herein are cleavage-inducing tiny RNAS (cityRNAs) which are capable of inducing AGO3 to slice nucleic acids. This powerful tool can be used in a variety of applications.

### CROSS-REFERENCE TO RELATED APPLICATIONS

This application claims benefit of U.S. Provisional Application No. 63/046,024 filed Jun. 30, 2020, and U.S. Provisional Application No. 63/110,405 filed Nov. 6, 2020, each of which is incorporated herein by reference in its entirety.

### GOVERNMENT SUPPORT CLAUSE

This invention was made with government support under grants R01 GM124320 and R01 GM138997 awarded by the National Institutes of Health. The government has certain rights in the invention.

### BACKGROUND

MicroRNAs (miRNAs) are small noncoding RNAs that control gene expression post-transcriptionally (Kozomara 2019; Bartel 2018). Their sequences differ, but their lengths generally fall within a range of 20˜23 nucleotides because the precursor miRNAs are processed by Dicer, which is a molecular ruler that generates size-specific miRNA duplexes (Zhang 2004; Macrae 2006). After those duplexes are loaded into AGOs, one of the two strands is ejected while the remaining strand (guide strand) and the AGO form the RNA-induced silencing complex (RISC) (Nakanishi 2016). Therefore, the 2023-nucleotide length is the hallmark of intact miRNAs. This size definition has been exploited as the rationale for eliminating ˜18 nucleotide RN As when AGO-bound miRNAs are analyzed by next-generation RNA sequencing (RNAseq). However, RNAseq without a size exclusion reported a substantial number of ˜18-nucleotide RNAs bound to AGOs (Kuscu 2018; Gangras 2018; Kumar 2014). Such tiny guide RNAs (tyRNAs) are known to be abundant in extracellular vesicles of plants (Baldrich 2019), but little was previously known about their roles or biogenesis pathways. In mammals, the roles of tyRNAs have been even more enigmatic.

In 2004, two groups reported that only AGO2 showed the guide-dependent target cleavage in vitro (Liu 2004; Meister 2004). Since then, AGO1, AGO3, and AGO4 were thought to be deficient in RNA cleavage, even though AGO3 shares the same catalytic tetrad with AGO2. Recently, it was revealed that specific miRNAs such as 23-nucleotide miR-20a make AGO3 a slicer, but the activity was much lower than that of AGO2 (Park 2017). Therefore, it was unknown until now that AGO3 is capable of becoming a highly competent slicer as well.

What is needed in the art are AGO3 complexes and corresponding guide RNAs which are capable of interacting with target nucleic acids. This can be used for a variety of applications, including treating and preventing disease, and diagnosis of disease or other disorders.

### SUMMARY

Disclosed herein is a method of regulating a target nucleic acid using an Argonaute-3 (AGO3) molecule, wherein the AGO3 functions as a slicer of the target nucleic acid, the method comprising: (a) preparing or isolating a double-stranded RNA molecule, wherein one of the strands comprises sufficient complementarity to hybridize with the target mRNA, wherein said double stranded RNA molecule comprises a cleavage-inducing tyRNA (cityRNA) of 12-16 nucleotides in length; (h) exposing the double-stranded RNA molecule to an RNA induced silencing complex (RISC) comprising AGO3 under conditions which allow for loading of the double-stranded RNA molecule into RISC; and (c), exposing the AGO3 associated RISC loaded with cityRNA to the target nucleic acid, thereby allowing AGO3-associated RISC to modify the target nucleic acid.

Also disclosed herein is a single- or double-stranded non-naturally occurring cleavage-inducing tyRNA (cityRNA) of 12-16 nucleotides in length, wherein the cityRNA is capable of activating slicing of AGO3. As described above, this cityRNA can be 12, 13, 14, 15, or 16 nucleotides in length. The cityRNA can be designed based on the intended target molecule. The cityRNA can be introduced to AGO3, either separately or as part of a double-stranded nucleic acid, which will be processed and introduced to the target nucleic acid by RISC, One of skill in the art will understand how to make such a molecule.

Further disclosed herein is a kit, wherein the kit comprises at least one cityRNA molecule. The cityRNA can be 14 nucleotides in length. The kit can further comprise an AGO3 molecule, as well as all or part of RISC, such as proteins that are associated therewith. The kit can also include other components which can be used in the methods disclosed herein. For example, the kit can comprise components suitable for AGO3 and the double stranded nucleic acid to form a complex.

Disclosed herein is a method of recruiting an AGO3 polypeptide to a target nucleic acid, the method comprising combining the AGO3 polypeptide with a double-stranded RNA comprising a cityRNA, wherein the cityRNA is 12-16 nucleotides in length. This can be used as a method of detecting a target nucleic acid. For example, the cityRNA, or any part of the AGO3 or RISC can comprise a detectable label. The detectable label can be a fluorescent dye or a radiolabel. The target nucleic acid can encode disease marker sequences, a disorder marker sequence, or an infectious agent sequence. The method can be carried out in a subject to diagnose or treat a disease or disorder.

Further disclosed is a method of identifying an RNA binding polypeptide comprising binding to a target nucleic acid sequence in an RNA molecule a complex comprising an AGO3 polypeptide and a cityRNA, wherein the cityRNA is 12-16 nucleotides in length, such that the AGO3 polypeptide: cityRNA complex binds stably to the target nucleic acid sequence; isolating the AGO3 polypeptide: cityRNA complex bound to the target nucleic acid sequence, and detecting polypeptides bound to the complex comprising the target nucleic acid binding sequence.

Disclosed herein is a method of determining a cleavage-inducing tyRNA (cityRNA), the method comprising exposing an AGO3 polypeptide to an array of potential cityRNAs, wherein said cityRNAs are about 12-16 nucleotides in length, and determining which of the array of potential cityRNAs are capable of forming a complex with AGO3. After it is determined that a cityRNA and an AGO3 have formed a complex, one can further determine whether said complex is capable of cleaving an RNA or DNA molecule.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIGS. 1A-1E show 14-nt miR-20a brings out the slicing activity of AGO3. (1A) Guide and target. RNAs used in B-D. The 60-nt target RNAs were cap-labeled. (1B-1C) In vitro cleavage assay by AGO2 and AGO3 with different lengths of miR-20a. Top: target cleavage percentages. Data are shown as mean (bar) and individual biological replicates (dots). Bottom: relative target cleavage (fold) with each guide against 23 nt. Data are shown as Mean+SD. (1D) Time-course assay of AGO2 and AGO3 with the 14- or 23-nt miR-20a. Data are shown as Mean±SD. (1E) In vitro target cleavage using AGO2 and AGO3 with the intact miRNAs or 14-nt tyRNAs. The number in parentheses indicates the relative target cleavage (fold increase) of each 14-nt tyRNA against the cognate intact miRNA. Data are shown as Mean±SD. *P<0.05; **P<0.01; *** P<0.001; **** P<0.0001; ns, not significant (Student's t test). SEQ ID NOS: 1-9 are shown in 1A (sequentially, from top to bottom).

FIG. 2 shows the purity of recombinant AGO proteins. Recombinant proteins of four human AGOs and FLAG-tagged AGO3 used in cleavage assays were analyzed on an SDS-PAGE.

FIG. 3 shows 14-nt miR-20a converts AGO3 to a highly competitive slicer. Representative images of in vitro cleavage assay using AGO2 and AGO3 with different lengths of miR-20a. The reactions were resolved on a 16% denaturing gel.

FIG. 4 shows 14- and 23-nt miR-20a variants. Left side: 14ss is identical to the 14-nt miR-20a in FIG. 1A. p14ss is identical to 14ss, except for the 5′-end radiolabeling (yellow circle). 14md is identical to 14ss, except for nucleotide modifications (blue: 2′-OMe, Green: 2′-F, red s: Phosphorothionate). Middle: 23ss is identical to the 23-nt miR-20a in FIG. 1A. p23ss is identical to 23ss, except for the 5′-end radiolabeling. Right side: 23ds is composed of a 23ss (top strand) and a passenger strand (bottom), the latter of which lacks a 5′ monophosphate group so AGOs load the guide strand (i.e., 23ss), p23ds is identical to 23ds, except for the 5′-end radiolabeling. SEQ ID NOS: 10-18 are depicted, in order from left to tight, top to bottom.

FIGS. 5A-5C show loading of 14- or 23-nt guide RNAs. (5A) A representative image of the in vitro guide RNA loading assay. 1 μM recombinant AGO2 and AGO3 were loaded with either 0.1, 1, 5, 10, 20, 50, 100, or 200 nM 14- or 23-nt 5′ end-labeled miR-20a and immobilized on nitrocellulose membrane. miR-20a did not bind to the membrane without pre-incubation with AGO. (5B to 5C) The in vitro loading assay of AGO2 (5B) and AGO3 (5C) was triplicated. The relative 14-nt miR-20a loading against 23-nt was plotted on the concentration of miR-20a.

FIG. 6 shows kinetics of target cleavage by AGO2 and AGO3. A representative image of an in vitro time-course target cleavage assay. Recombinant AGO2 and AGO3 were programmed with the 14- or 23-nt miR-20a and incubated with the cap-labeled 60-nt target RNA.

FIGS. 7A-7B show cityRNAs activate mainly AGO3 and confer AGO2-like target recognition. (7A) in vitro target cleavage of four human AGOs with either 14- or 23-nt miR-20a. (7B) In vitro target cleavage of AGO2 and AGO3 with 14- or 23-nt miR-20a. The reactions were resolved on 16% denaturing gel alongside base-hydrolyzed 45-nt polyuridine RNA.

FIGS. 8A-8C show some of the tyRNAs activate AGO3 for RNA cleavage. (8A) Guide and target RNAs. The sequence of eight different guide RNAs and the corresponding target RNAs used for in vitro cleavage assay in FIG. 1E. (8B) In vitro target cleavage using AGO2 or AGO3 with the 14-nt or intact let-7a, miR-16, and miR-19b. (8C) In vitro target cleavage using AGO2 or AGO3 with the 14-nt or intact miR-17, miR-18a, miR-19a, miR-27a, and miR-92a. The intact target that formed a very stable duplex with the 23-nt miR-92 is marked with *. Sequences in FIG. 8A are represented by SEQ BD NOS: 19-42, sequentially from top to bottom.

FIGS. 9A-9I show expression of FLAG-AGOs in HEK293T cells and two different types of in vitro cleavage assays. (9A) A representative image of western blot. FLAG-AGO2, -AGO3, and 5-AGO3 (E638A) were expressed in HEK293T cells and detected with anti-FLAG antibody. (9B to 9D) Representative images of in vitro cleavage assay using FLAG-AGO2 (9B), -AGO3 (9C), and -AGO3 (E638A) (9D). All lysates were adjusted by western blots of (A) and incubated with 14-nt single-stranded miR-20a or 23-nt siRNA-like duplex of miR-20a for RISC assembly. 5′ cap-labeled target RNAs were used for target cleavage. (9E) in vitro cleavage assay of recombinant AGO3 programmed with the unmodified and modified 14- and 23-nt single-stranded miR-20a(s). Statistical analyses: mean and individual biological replicates. P<0.05. (9F to 9G) Representative images of western blot. FLAG-AGO2 (9F) and -AGO3 (9G) 5 were expressed in HEK293T cells transfected with the 14-nt unmodified single-stranded miR-20a, the 14-nt modified single-stranded miR-20a, or the 23-nt siRNA-like duplex of miR-20a. (9H to 9I) Representative images of in vitro cleavage assay using FLAG-AGO2 (9H) and -AGO3 (9I). Both AGOs were programmed in HEK293T cells. All the lysates were normalized by the western blot analysis (9F to 9G) and incubated with a 5′ cap-labeled target RNAs for target cleavage. The reactions were resolved on a 16% polyacrylamide gel.

FIGS. 10A-10B show AGO3 becomes a competitive slicer. Time-course assay of AGO2 and AGO3 with the 14- or 23-nt miR-20a (10A) and the 14- or 21-nt let-7a (10B).

FIGS. 11A-11D show a comparison of the RISC structures between AGO3 and AGO2. 11A-11B. (right) Crystal structures of AGO3 (11A) and AGO2 (11B) in complex with guide (red). Their catalytic tetrads are shown in box. (left) Cross sections of AGO3 (11A) and AGO2 (11B) along the nucleic acid-binding channel. The channel of AGO3 is wider than that of AGO2. AGO3 Specific Insertion (3 SI) and the corresponding part of AGO2 are highlighted with orange circles. The connection between the N and L1 domain is highlighted with arrow heads (orange). 11C-11D. Interaction between the N and L1 domains of AGO3 (11C) and AGO2 (11D), The van der Waals radii of nonpolar residues involved in the N-L1 interaction are depicted as dots. AGO3 Specific Insertion (3 SI) is drawn as dotted lines (orange).

FIGS. 12A-12E show the difference in the activation mechanism between AGO2 and AGO3. (12A) Target RNAs of miR-20a including both 5′ and 3′ flanking regions (intact) and lacking the 5′ flanking region (Δ5′) and the 3′ flanking one (Δ3′). (12B-12C) In vitro cleavage of the three targets shown in a by AGO3 (12B) and AGO2 (12C), 12D-12E, Activation models of AGO2 (12D) and AGO3 (12E), FIG. 12A shows sequences represented by SEQ ID NOS: 43-48, sequentially from top to bottom.

FIG. 13 shows length distribution of AGO-bound guide RNAs.

FIGS. 14A-14F show impact of nucleotides common between miR-20a and let-7a. (14A) Nucleotides shared between miR-20a and let-7a are colored in red. (14B) Design of single-nucleotide mutation on miR-20a. (14C) Design of the fully complementary target (blue) of a 14-nt miR-20a (A3C), (14D) Nucleotide incorporation into miR-16. (14E) Replacement of the common nucleotides between miR-16 and miR-19b (green) with the corresponding ones of miR-20a and let-7a (red), (14F) Replacement of the nucleotides found in neither miR-20a nor let-7a (cyan) with the corresponding ones of miR-20a and let-7a (red), Sequences in FIGS. 14A-14F are represented by SEQ ID NOS: 49-60, sequentially from top to bottom.

FIGS. 15A-15C show purification of a homogeneous RISC. (15A) Schematic of the modified Arpón method. (15B) SDS-PAGE analysis of a homogeneous AGO2 programmed with the 21-nt let-7a. AGO2 pre-occupied by insect cell-endogenous small RNA was removed in Unbound fraction (see a). (15C), In vitro RNA cleavage assay using Elution fraction of 15B. (see a). Although no let-7a was added to Elution fraction, almost all target was cleaved, demonstrating the success in purification of homogeneous AGO2-let-7a complex.

FIG. 16 shows design of single nucleotide mismatch, A wild-type 14-nt miR-20a (red) and a 60-nt cap-labeled target RNA (blue) are base-paired through g2-g14. Single-nucleotide mismatches are black letters. Sequences are represented by SEQ ID NOS: 61-76, sequentially from top to bottom.

FIG. 17 shows design of target RNAs. 14-nt miR-20a (red) is base-paired with target RNA variants (blue): The 5′ end G and m7G of target RNAs are colored in cyan. Sequences are represented in SEQ ID NOS: 77-86, sequentially from top to bottom.

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 8, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA, In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes, A single-stranded oligonucleotide can exist as a linear molecule without any hydrogen-bonded nucleotides, or can fold three-dimensionally to form hydrogen bonds between individual nucleotides along the single stranded oligonucleotide.

The term “polynucleotide” refers to a single or double stranded polymer composed of nucleotide monomers. Polynucleotides can be any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated. RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. A polynucleotide is composed of a specific sequence of four nucleotide bases: adenine (A); cytosine (C); guanine (G); thymine (T); and uracil (U) for thymine (I) when the polynucleotide is RNA. Thus, the term “polynucleotide sequence” is the alphabetical representation of a polynucleotide molecule. In some embodiments, the polynucleotide is composed of nucleotide monomers of generally greater than 100 nucleotides in length and up to about 8,000 or more nucleotides in length.

The term “polypeptide” refers to a compound made up of a single chain of D- or L-amino acids or a mixture of D- and L-amino acids joined by peptide bonds.

The term “complementary” or “complementarity” refers to the topological compatibility or matching together of interacting surfaces of two molecules (e.g., a probe molecule and its target, particularly a DNA guide molecule and a target RNA molecule). Thus, the two molecules (e.g., target and its probe) can be described as complementary, and furthermore, the contact surface characteristics are complementary to each other. In the case of nucleotides or polynucleotides (e.g., DNA or RNA), the two molecules are complementary if they have sufficiently compatible nucleotide base-pairs such that the two molecules can hybridize. The term “complementary,” as it relates to nucleotide molecules (e.g., nucleotides, oligonucleotides, polynucleotides, modified nucleotides, etc.), is intended to include two or more nucleotide molecules which have 100% complementarity (e.g., each nucleotide in a sequence of one molecule is the nucleotide base-pair complement of an adjacent nucleotide in a sequence of the second molecule, in sequential order) as well as two or more nucleotide molecules which have less than 100% complementarity but which hybridize under the conditions of the methods disclosed herein.

The term “hybridization” or “hybridizes” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally-occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements ((e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters, Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p, 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e. a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software, Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPS) by identifying short words of length W in the query sequence, which either match or satisfy some positive-valued threshold score T when aligned with a word of the same length in a database sequence. T is referred to as the neighborhood word score threshold (Altschul et al, (1990)J. Mol. Biol. 215:403-410), These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4 and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example; a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

A polynucleotide sequence is “heterologous” to a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from naturally occurring allelic variants.

The phrase “selectively (or specifically) hybridizes to” refers to the binding, duplexing, or hybridizing of a molecule only to a particular nucleotide sequence with a, higher affinity, e.g., under more stringent conditions, than to other nucleotide sequences (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as follows: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et at One of skill will recognize that these values can be appropriately adjusted to determine corresponding identity of proteins encoded by two nucleotide sequences by taking into account codon degeneracy, amino acid similarity, reading frame positioning and the like. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine-valine; aspartic acid-glutamic acid, and asparagine-glutamine.

Methods

More than 2,000 microRNAs (miRNAs) have been reported as of 2019 in humans (Kozomora 2018), miRNAs are varied in sequence, but their lengths fall within a range of 19˜23 nucleotides (nt) because precursor miRNAs are processed by Dicer which is a molecular ruler that generates size-specific miRNA duplexes (Zhang 2004; Macrae 2006; MacRae 2007). After those duplexes are loaded into Argonaute proteins (AGOs), one of the two strands is ejected while the remaining strand (guide strand) and AGO form the RNA-induced silencing complex (RISC) (Nakanishi 2016; Meister 2013; Wilson 2013; Jinek 2009). Therefore, the 19˜23-nucleotide length of small RNAs is the hallmark of mature miRNAs. This size definition was exploited to eliminate ˜18-nucleotide RNAs during sample preparation or analysis in most of the early next generation RNA sequencing (RNAseq) of miRNAs. On the other hand, RNAseq without RNA elimination found a substantial number of 10˜18-nucleotide tiny RNAs (tyRNAs) bound to AGOs (Gangras 2017; Kuscu 2018; Baldrich 2019). Although some of tyRNAs are known to regulate gene expression similarly to mature miRNAs, little was known about whether tyRNAs play any specialized role.

Subsequently, in vitro studies revealed that 14˜15-nucleotide tyRNAs derived from specific miRNAs conferred a competitive slicing activity on AGO3. These tyRNAs are referred to as cleavage-inducing tyRNAs (cityRNAs). Furthermore, the RNAseq analyses showed that quite a few tyRNAs were bound to AGO3. It appears that many tyRNAs serve as cityRNAs. To date, many studies have focused on AGO2 based on the previous reports that only AGO2 can cleave RNAs (Wittrup 2015; Kannan 2018) and that the gene is essential (Cheloufi 2010). Since the mutation or deletion on the AGO2 gene is too fatal to cure with current treatment, infants with the genetic disease would not survive. Meanwhile, it has been reported that patients who suffer from neurological disease have mutations and deletions within the AGO3 gene (Tokita 2014), suggesting a possibility that AGO3 is not an essential gene for its survival but critical for normal body growth and neural development.

Disclosed herein is the finding that AGO3 becomes a competitive slicer of AGO2 when loaded with tiny RNAs (tyRNAs), which are smaller in size than those to trigger slicer activity in AGO2. For example, this takes place when miR-20a has the 3′ 8˜9 nucleotides deleted. Surprisingly, even a 14-nucleotide tyRNA of let-7a converted AGO3 to a slicer. In contrast, AGO2 drastically decreased the slicing activity when loaded with those tyRNAs. These findings demonstrate that AGO2 and AGO3 have a different optimum length of guide RNA for slicing activity.

Specifically disclosed herein is a method of regulating a target nucleic acid using an ARGONAUTE-3 (AGO3) molecule, wherein the AGO3 functions as a slicer of the target nucleic acid, the method comprising: (a) preparing or isolating a double-stranded RNA molecule, wherein one of the strands comprises sufficient complementarity to hybridize with the target mRNA, wherein said double stranded RNA molecule comprises a cleavage-inducing tyRNA (cityRNA) of 12-16 nucleotides in length; (b) exposing the double-stranded RNA molecule to an RNA induced silencing complex (RISC) comprising AGO3 under conditions which allow for loading of the double-stranded RNA molecule into RISC; and (c), exposing the AGO3 associated RISC loaded with cityRNA to the target nucleic acid, thereby allowing AGO3-associated RISC to modify the target nucleic acid.

The cityRNA used with the methods disclosed herein can be 12, 13, 14, 15, or 16 nucleotides in length. In one specific embodiment, the cityRNA molecule is 14 nucleotides in length. This cleavage can result in RNA silencing, for example, and can be used to treat or prevent a variety of diseases and disorders known to those of skill in the art.

In one embodiment, the target nucleic acid sequence is from a mammal. In one embodiment, the target nucleic acid sequence is from a human. The target nucleic acid sequence can be RNA or DNA. In a specific example, the target RNA can be mRNA. The cityRNA disclosed herein can be 10, 20, 30, 40, 50, 60, 70, 80, 90, or 100% identical to the target nucleic acid, or any amount below or between these amounts. Viewed another way, the cityRNA can have 1, 2, 3, 4, or 5 mismatches within the complementary region, or can be completely complementary (no mismatches). The target nucleic acid can be longer than the cityRNA. For example, it can be considerably longer, as in part of an mRNA that encodes a protein. In this case, the cityRNA can hybridize with the target nucleic acid, but there can be substantial parts of the target nucleic acid that do not hybridize with the cityRNA.

In one embodiment, the cityRNA can have at least one chemically modified nucleotide. These modified nucleotides may confer increased stability, decreased off-target effects, and/or reduced toxicity, as compared to a ssDNA not having the chemically modified nucleotide. They can also facilitate detection.

In some embodiments, the at least one chemically modified nucleotide comprises a chemically modified nucleobase, a chemically modified ribose, a chemically modified phosphodiester linkage, or a combination thereof.

In one embodiment, the chemically modified nucleobase is selected from 5-formylcytidine (5fC), 5-methylcytidine (5meC), 5-methoxycytidine (5moC), 5-hydroxycytidine (5hoC), 5-hydroxymethylcytidine (5hmC), 5-formyluridine (5fU), 5-methyluridine (5-meU), 5-methoxyuridine (5moU), 5-carboxymethylesteruridine (5camU), pseudouridine (Ψ), N1-methylpseudouridine (me1Ψ), N6-methyladenosine (me6A), or thienoguanosine (thG).

In one embodiment, the chemically modified ribose is selected from 2′-O-methyl (2′-O-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl (2′-O-ME), 2′-O-Allyl, 2′-O-Ethylamine, 2′-O-Cyanoethyl, Locked nucleic acid (LAN), Methylene-cLAN, N-MeO-amino BNA, or N-MeO-aminooxy BNA.

In one embodiment, the chemically modified phosphodiester linkage is selected from Phosphorothioate (PS), Boranophosphate, phosphodithioate (PS2), 3′,5′-amide, N3′-phosphoramidate (NP), Phosphodiester (PO), or 2′,5′-phosphodiester (2′,5′-PO).

In some embodiments, the Argonaute-3 (AGO3) polypeptide used with the methods disclosed herein is from a yeast. In some embodiments, the Argonaute polypeptide is from Vanderwaltozyma polyspora (also known as Kluyveromyces polysporus). Additional non-limiting examples of yeast Argonaute polypeptides can be from additional yeast species of the genus Kluyveromyces: K. aestuari, K. africanus, K. bacillisporus, K. blattae, K. dobzhanskii, K. hubeiensis, K. lactis, K. lodderae, K. marxianus, K. nonfermentans, K. piceae, K. sinensis, K. thermotolerans, K. waltii, K. wickerhamii, or K. yarrowi. Additional non-limiting examples of yeast Argonaute polypeptides can be from Yarrowia lipolytica, Pichia pastori, Candida vulgaris, Saccharomyces castellii, or Schizosaccharomyces pombe.

In some embodiments, the AGO3 polypeptide used with the methods disclosed herein is from a eukaryote. In some embodiments, the AGO3 polypeptide is from a mammal. In some embodiments, the AGO3 polypeptide is from a primate. In some embodiments, the AGO3 polypeptide is from a human.

In some embodiments, the AGO3 polypeptide is a full length AGO3 polypeptide. In some embodiments, the AGO3 polypeptide comprises a portion of the AGO3 protein.

In one embodiment, the AGO3 polypeptide is a wild-type sequence. In one embodiment, the AGO3 polypeptide is a sequence with at least one mutation. In one embodiment, the AGO3 polypeptide comprises an amino acid sequence that is different from a naturally-occurring AGO3 polypeptide.

In some embodiments, the system and methods may comprise additional polypeptides in addition to the AGO3 polypeptide. For example, additional components of the RISC complex may be present.

In the natural process of RNAi and gene silencing using RISC, long double-stranded RNAs are cleaved by the RNase III family member, Dicer, into nucleotides (nt) fragments with 5′ phosphorylated ends and 2-nt unpaired and unphosphorylated 3′ ends. AGO-3 then incorporates the guide strand into the RNA Interference Specificity Complex (RISC), while the passenger strand is released. The conditions which allow for loading of the double-stranded RNA molecule into RISC include the degradation of the passenger strand, thereby forming the cityRNA.

RISC uses the guide strand to find the target nucleic acid that has a complementary sequence leading to the endonucleolytic cleavage of the target mRNA. Therefore, the double-stranded RNA disclosed herein can be cleaved before exposure to RISC. Alternatively, only the cityRNA can be introduced to the RISC molecule.

Once RISC has been loaded with the cityRNA, it can be used for a variety of purposes. For example, it is known that cityRNA can slice, or cleave, the target nucleic acid. This can effectively “silence” the target nucleic acid. This can be used to treat a variety of diseases and disorders. One can imagine that any time that a nucleic acid should be destroyed or silenced, the method disclosed herein can be employed. For example, dysfunctional gene expression can be modified including, but not limited to, infectious diseases, particularly viral, bacterial or protozoal diseases. The methods disclosed herein can also be used to treat cancer.

The target nucleic acid may be a reporter gene, a pathogen-associated gene, e.g. a viral, protozoal or bacterial gene, or an endogenous gene, e.g. an endogenous mammalian, particularly human gene. The endogenous gene may be associated with a disorder, particularly with a hyperproliferative disorder, e.g. cancer, or with a metabolic disorder, e.g. a disorder associated with carbohydrate, energy, lipid, nucleotide, or amino acid metabolism or a disorder associated with the biosynthesis or metabolism of glycans, polyketides and nonribosomal peptides, cofactors and vitamins or secondary metabolites, with the biodegradation of xenobiotics or with a neurodegenerative disorder such as Alzheimer, Parkinson, Huntington, ALS, MS etc. Thus, the present invention is suitable for the manufacture of reagents, diagnostics and therapeutics.

For pharmaceutical applications, the invention provides also a pharmaceutical composition comprising as an active agent at least one city RNA molecule as described herein, or a precursor thereof or a DNA molecule encoding the cityRNA molecule or the precursor and a pharmaceutical carrier. The composition may be used for diagnostic and therapeutic applications in human medicine or in veterinary medicine.

For diagnostic or therapeutic applications the composition may be in form of a solution, e.g. an injectable solution, a cream, ointment, tablet, suspension or the like. The composition may be administered in any suitable way, e.g. by injection, by oral, topical, nasal, rectal application etc. The carrier may be any suitable pharmaceutical carrier. Preferably, a carrier is used of increasing the efficacy of RNA molecules to enter the target cells. Suitable examples of such carriers are liposomes, particularly cationic liposomes.

A further aspect of the invention relates to the modulating of a target gene specific silencing activity in a cell, an organism or a cell-free system, wherein the activity of at least one polypeptide of the gene silencing machinery is selectively modulated, e.g. increased and/or suppressed. By means of this selective activity increase and/or suppression, the efficacy of target nucleic acid specific silencing may be considerably increased. Thus, administration of double stranded molecules directed to the mRNA of a target gene, organism or a cell-free system (as indicated above) may be more effective.

The gene-specific silencing can comprise transcriptional gene silencing (TGS) activity or a post-transcriptional gene silencing (PTGS) activity. PTGS includes translational attenuation and/or RNA interference. Three phenotypically different but mechanistically similar forms of RNAi, co-suppression or PTGS in plants, quelling in fungi, and RNAi in the animal kingdom, have been described. The cityRNA can comprise a siRNA, shRNA or a miRNA molecule.

Also disclosed herein is a method of recruiting an AGO3 polypeptide to a target nucleic acid, the method comprising combining the AGO3 polypeptide with a double-stranded RNA comprising a cityRNA, wherein the cityRNA is 12-16 nucleotides in length. This can be used as a method of detecting a target nucleic acid. For example, the cityRNA, or any part of the AGO3 or RISC can comprise a detectable label. The detectable label can be a fluorescent dye or a radiolabel. The target nucleic acid can encode disease marker sequences, a disorder marker sequence, or an infectious agent sequence. The method can be carried out in a subject to diagnose or treat a disease or disorder.

Further disclosed is a method of identifying an RNA binding polypeptide comprising binding to a target nucleic acid sequence in an RNA molecule a complex comprising an AGO3 polypeptide and a cityRNA, wherein the cityRNA is 12-16 nucleotides in length, such that the AGO3 polypeptide: cityRNA complex binds stably to the target nucleic acid sequence; isolating the AGO3 polypeptide: cityRNA complex bound to the target nucleic acid sequence, and detecting polypeptides bound to the complex comprising the target nucleic acid binding sequence.

Similarly, disclosed herein is a method of determining a cleavage-inducing tyRNA (cityRNA), the method comprising exposing an AGO3 polypeptide to an array of potential cityRNAs, wherein said cityRNAs are about 12-16 nucleotides in length, and determining which of the array of potential city RNAs are capable of forming a complex with AGO3. After it is determined that a cityRNA and an AGO3 have formed a complex, one can further determine whether said complex is capable of cleaving an RNA or DNA molecule.

Binding of the AGO3:cityRNA complex to the target RNA or DNA molecule is significantly faster than AGO2. For example, it can be 5, 10, 15, 20, 25, 30, 35, 40, 45, 50, 55, 60, 65, 70, 75, 80, 85, 90, 95, 100, 150,200, 250, 300, 350, 400, 450, or 500 or more times faster than AGO2 binds to the target.

Compositions and Kits

Disclosed herein is a single- or double-stranded non-naturally occurring cleavage-inducing tyRNA (cityRNA) of 12-16 nucleotides in length, wherein the cityRNA is capable of activating slicing of AGO3. As described above, this cityRNA can be 12, 13, 14, 15, or 16 nucleotides in length. The cityRNA can be designed based on the intended target molecule. The cityRNA can be introduced to AGO3, either separately or as part of a double-stranded nucleic acid, which will be processed and introduced to the target nucleic acid by RISC. One of skill in the art will understand how to make such a molecule.

Also disclosed herein is a kit, wherein the kit comprises at least one cityRNA molecule. The cityRNA can be 14 nucleotides in length. The kit can further comprise an AGO3 molecule, as well as all or part of RISC, such as proteins that are associated therewith. The kit can also include other components which can be used in the methods disclosed herein. For example, the kit can comprise components suitable for AGO3 and the double stranded nucleic acid to form a complex.

### EXAMPLES

### Example 1: Human Argonaute2 and Argonaute3 are Catalytically Activated by Different Lengths of Guide RNA

Human Argonaute3 (AGO3) was recently revealed to become a slicer with a 23-nucleotide (nt) miR-20a, albeit showing much lower activity than Argonaute2 (AGO2). -nt 3′ end-shortened variants of let-7a, miR-27a, and specific miR-17-92 families were reported that make AGO3 an extremely competent slicer by an ˜82-fold increase in target cleavage. These RNAs, named cleavage-inducing tiny guide RNAs (cityRNAs), conversely lower the slicing activity of AGO2, demonstrating that AGO2 and AGO3 have different optimum guide lengths for target cleavage. FLAG-AGO3 loads transfected 14-nt single-stranded RNAs in HEK293T cells to form an active slicer. Disclosed herein is a model wherein the primary AGO slicer switches based on their guide length.

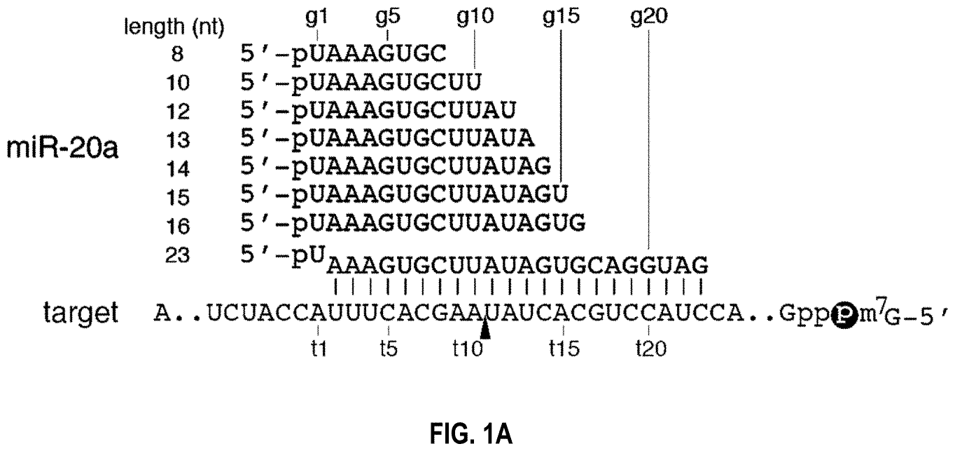

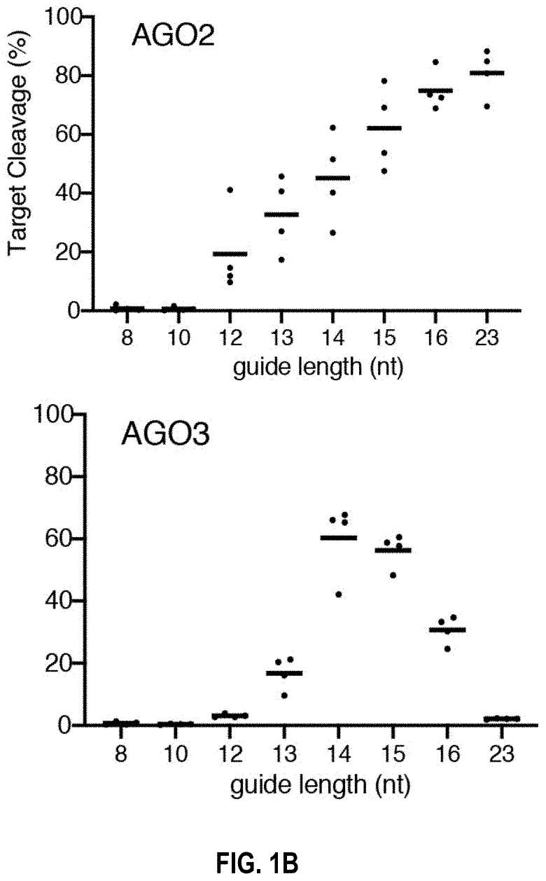

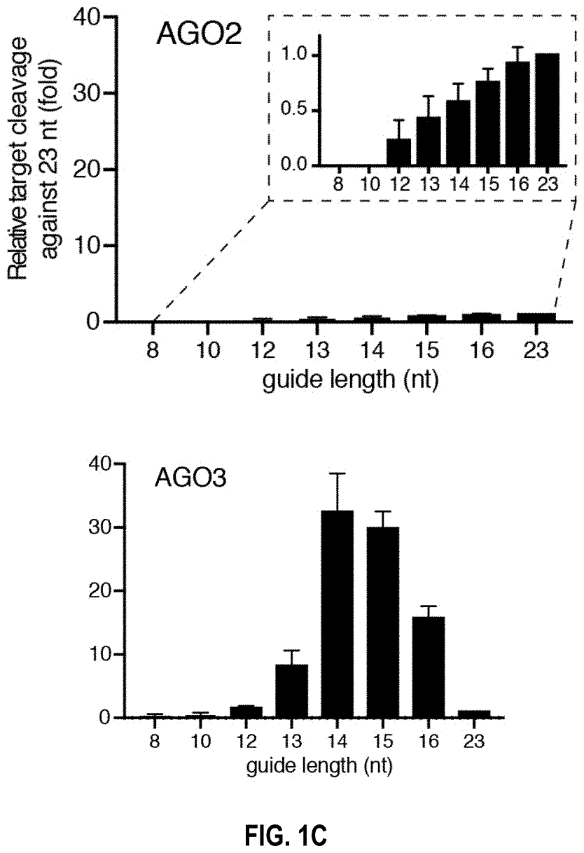

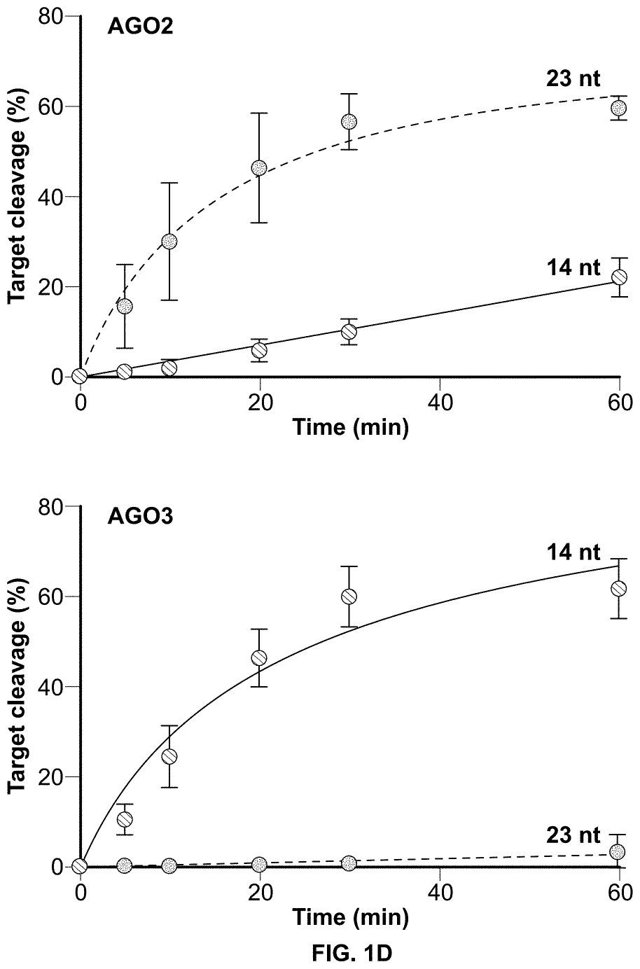

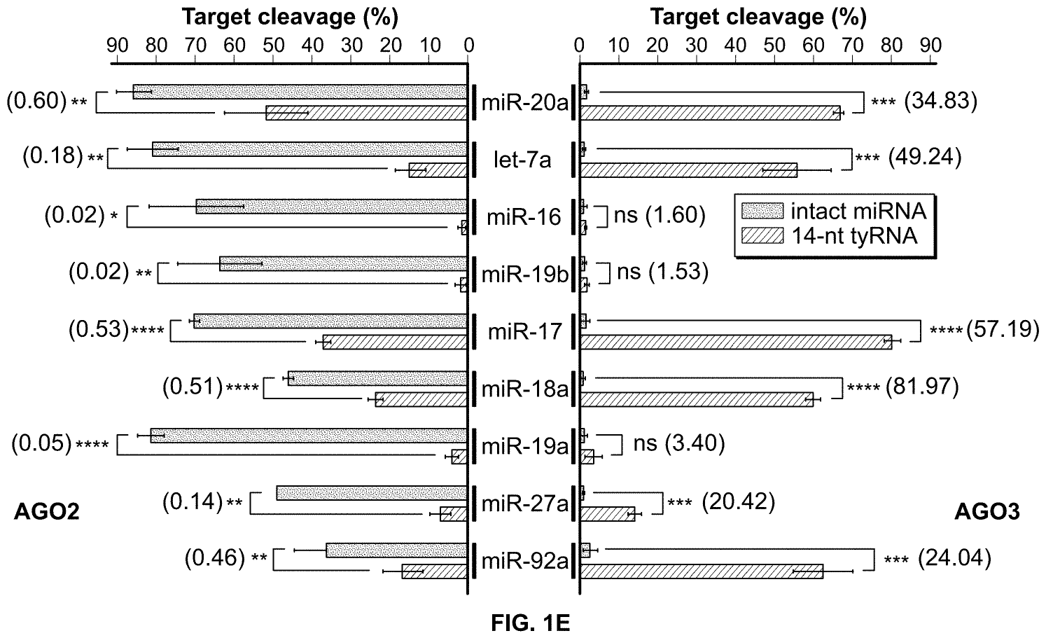

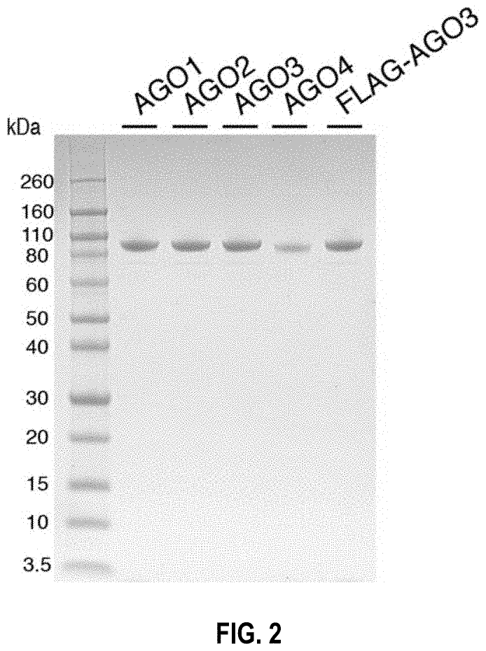

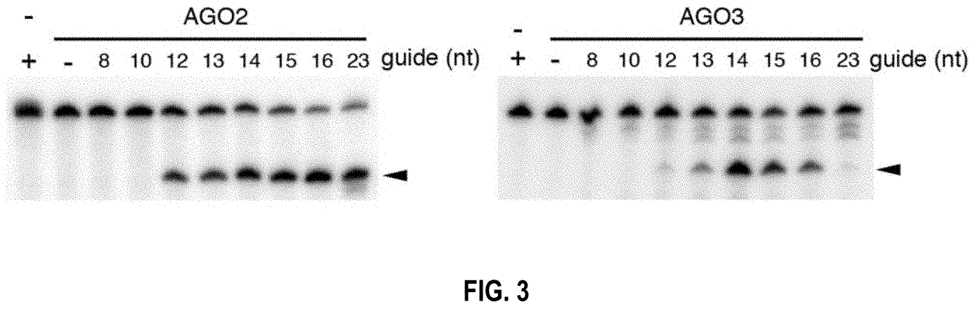

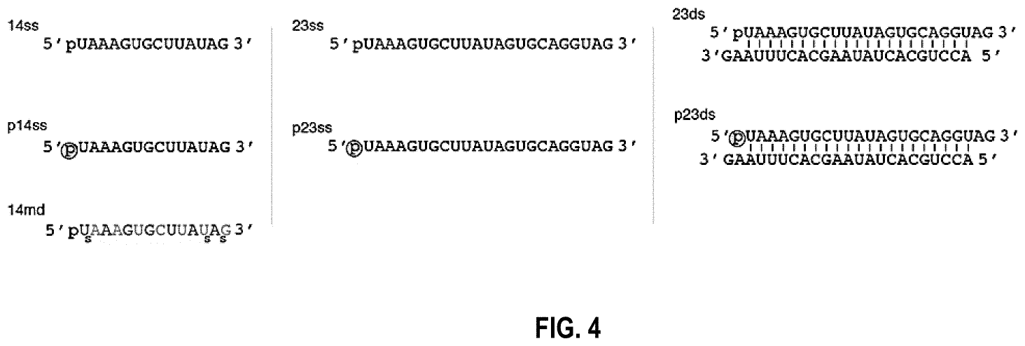

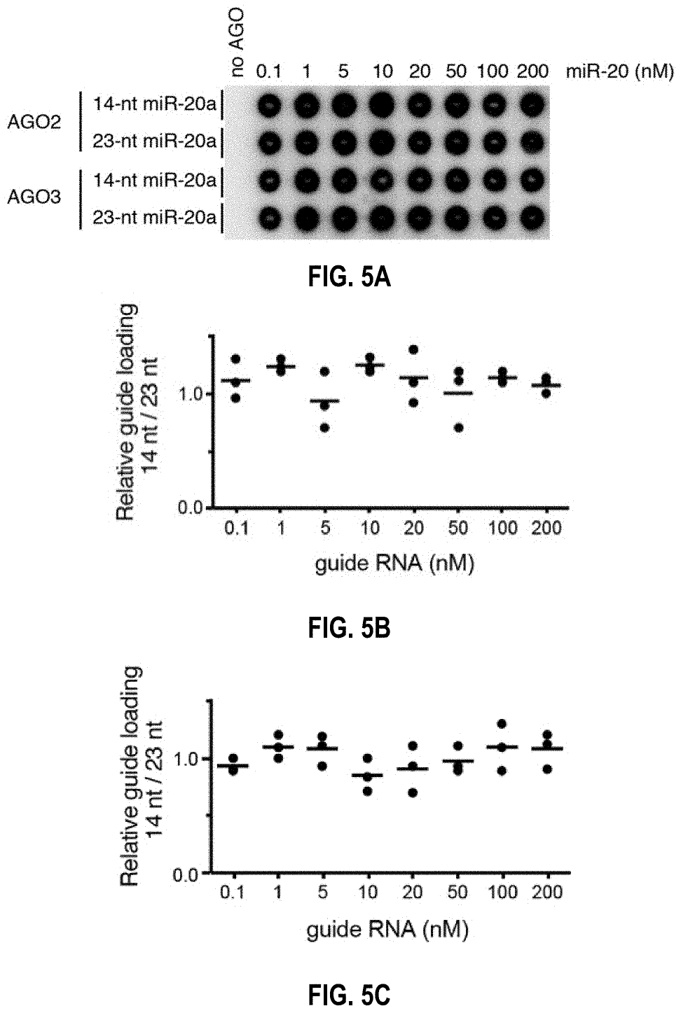

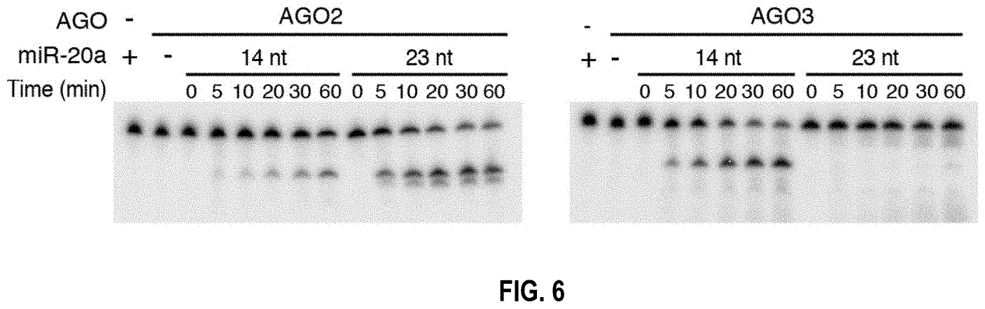

Recombinant AGO2 and AGO3 (FIG. 2) were pre-incubated with either of 8, 10, 12, 13, 14, 15, 16, or 23-nt single-stranded synthetic miR-20a whose 3′ 7˜15 nt are deleted, followed by addition of a cap-labeled target RNA (FIG. 1A) as previously reported (Dayeh 2018). While AGO2 reduced slicing activity with a shorter guide, AGO3 showed the highest cleavage activity with the 14-nt guide (FIG. 1B and FIG. 3). Notably, the slicing activity of AGO3 with the 14-nt miR-20a was about 30-fold higher than its 23-nt intact form (FIG. 1C), which resulted in AGO3 being a comparative slicer to AGO2. To examine whether the observed differences in target cleavage were attributed to the loading efficiency of different length guides, filter-binding assay's were performed where AGO2 and AGO3 were incubated with a 5′ end-labeled 14- or 23-nt single-stranded miR-20a (p14ss and p23ss, respectively in FIG. 4). As a result, both AGOs loaded the two guides similarly, regardless of their length (FIG. 5), indicating that the observed differences in the target cleavage were not due to the amount of mature RISC, but rather to the guide length. Supporting this, the kinetics of target cleavage with the 14- and 23-nt guide showed opposite trends between AGO2 and AGO3 (FIG. 1D, and FIG. 6) These results suggest that AGO2 and AGO3 have different optimum lengths of guide RNA for target cleavage.

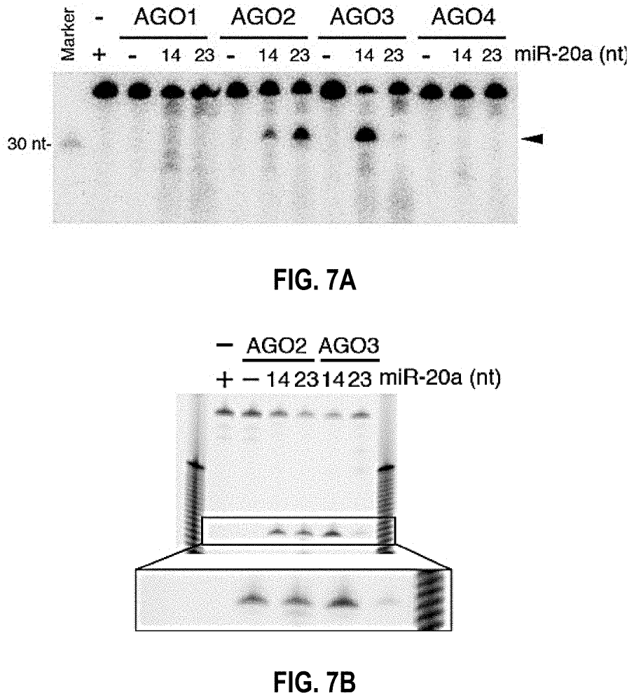

Previous structural and functional studies reported that AGO1 and AGO4 have a pseudo-catalytic tetrad (Nakanishi 2013: Faehnle 2013; Park 2019), which is the rationale for thinking that they lack slicing activity. It was tested whether the 14-nt miR-20a can activate these two AGOs. When programmed with the 14- or 23-nt miR-20a, neither showed slicing activity (FIG. 7A), suggesting that the 14-nt miR-20a mainly activates AGO3. Both AGO2 and AGO3 loaded with 14-nt miR-20a generated the same size of cleavage products when programmed with its intact length. This result demonstrates that AGO3 with the 14-nt miR-20a cleaves RNAs between two target nucleotide positions 10 and 11 (t10 and t11), that pair with guide nucleotide positions 10 and 11 (g10 and g11), respectively (FIG. 1A) Hereafter, tyRNAs capable of catalytically activating AGO3 are referred to as cleavage-inducing tyRNAs (cityRNAs).

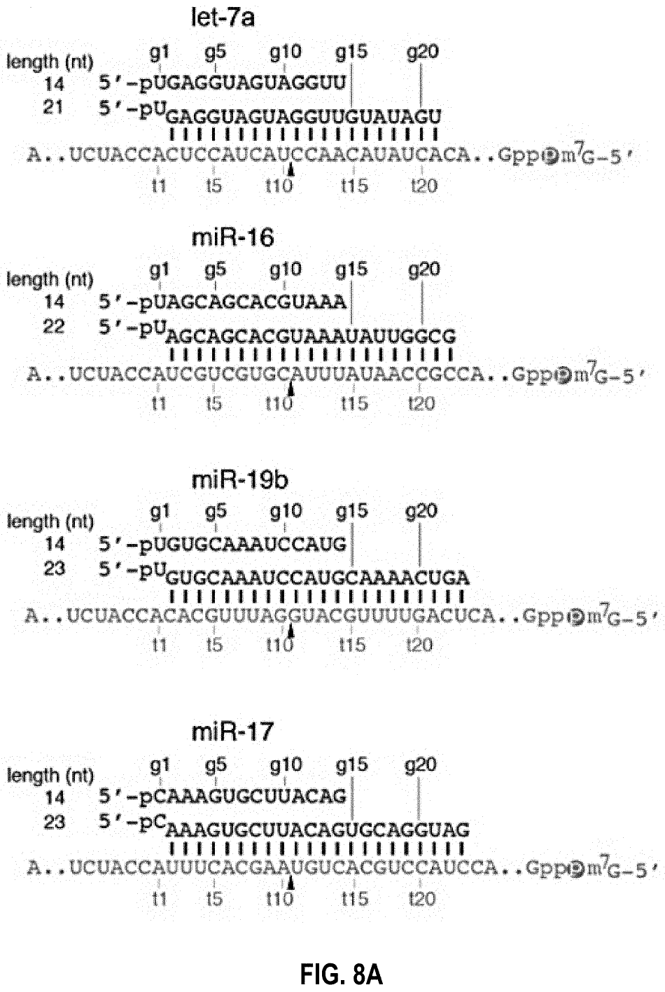

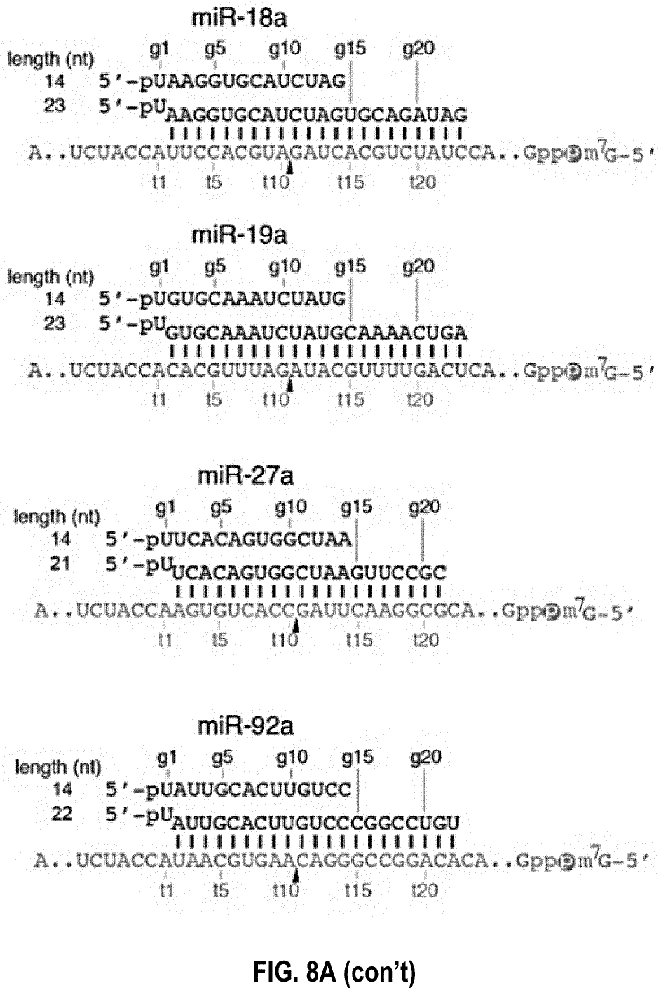

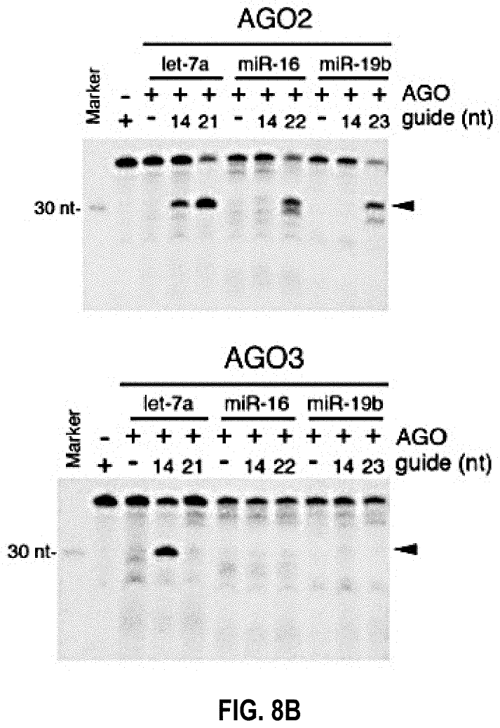

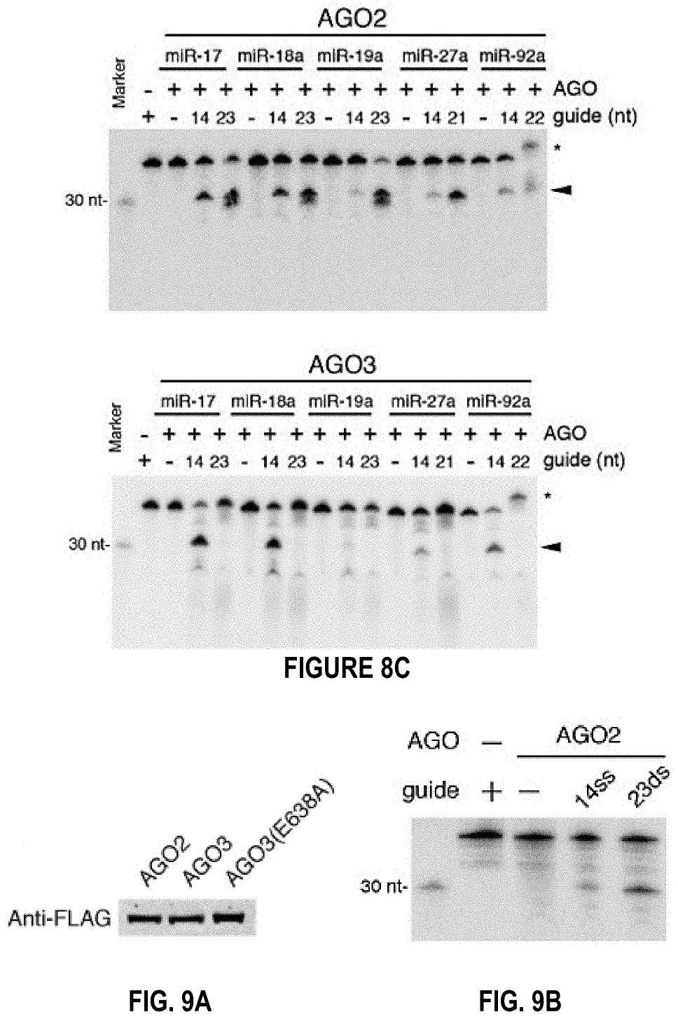

Intact miRNAs of let-7a, miR-16, and miR-19b are known to activate AGO2 but not AGO3 (Park 2017). To test whether their 14-nt tyRNAs serve as cityRNAs, recombinant AGO2 and AGO3 (FIG. 2) were programmed with either of their intact miRNA or tyRNA, and incubated with the cap-labeled target RNA. Loading of the tyRNAs drastically decreased or ruined the slicing activity of AGO2, compared to that of their intact form (FIG. 1E and FIG. 8A). In contrast, not the 14-nt miR-16 or miR-19b, but the 14-nt let-7a conferred extremely competent slicing activity on AGO3. To find more cityRNAs, 14-nt tyRNAs of miR-17, miR-18a, miR-19a, miR-27a, and miR-92a (FIG. 8A) were tested for in vitro target cleavage. Again, AGO2 reduced slicing activity with their 14-nt tyRNAs whereas AGO3 became a remarkably competent slicer when loaded with all except for the 14-nt miR-19a (FIG. 1E and FIG. 8C). These results indicate that not all tyRNAs can catalytically activate AGO3 and that some cityRNAs make AGO3 a superior slicer to AGO2.

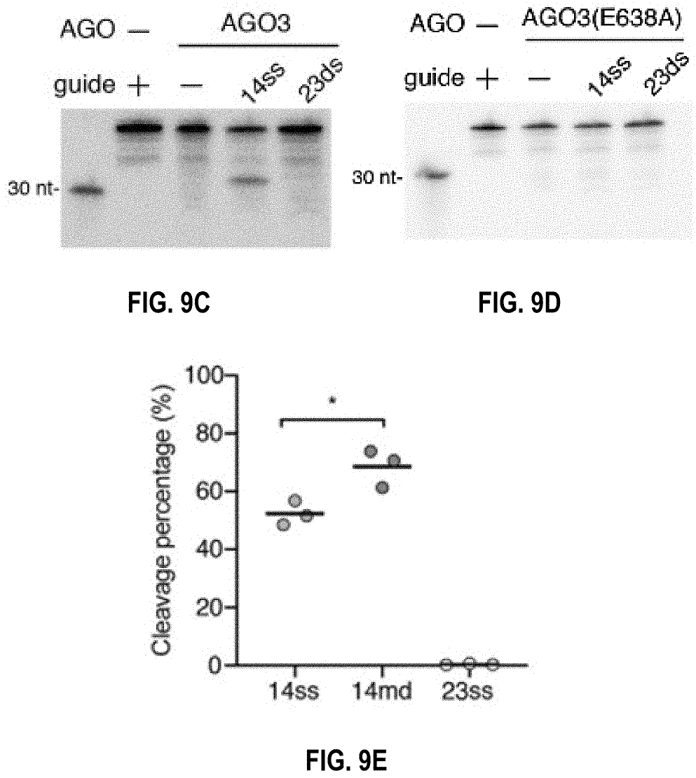

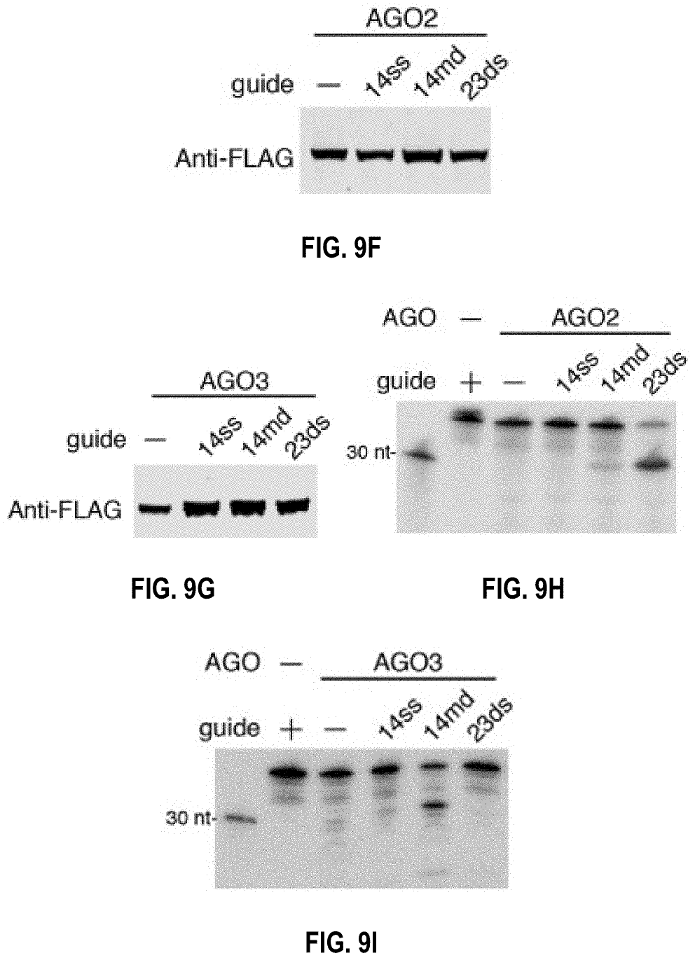

A potential pathway of tyRNA biogenesis is where preprocessed or degraded short RNAs are directly incorporated into AGOs to form the RISCs. Unlike miRNA duplexes, 14-nt RNAs are too short to form stable double-stranded RNAs (dsRNAs) at 37° C. Thus, it was thought that such short RNAs could be loaded as a single-stranded RNA (ssRNA) into AGOs. To test this idea, a RISC maturation assay was performed (Park 2019; Iwasaki 2018). Briefly, a 5′ end-labeled 14-nt single-stranded miR-20a (p14ss of FIG. 4) was added to the lysate of HEK293T cells expressing FLAG-AGO2 or FLAG-AGO3 (FIG. 9A), followed by immunoprecipitation with anti-FLAG beads. Then, AGO-bound RNAs were extracted and resolved on a native gel. In the positive control experiment, the 23-nt siRNA-like duplex of miR-20a (p23ds of FIG. 4) was used instead of p14ss. As expected, the provided 23-nt duplex was detected as its intact single strand in both AGO2 and AGO3, indicating that the siRNA-duplex underwent passenger ejection without being processed by any of the endogenous ribonucleases. Similarly, the intact 14-nt miR-20a was detected from both AGOs, demonstrating that AGO2 and AGO3 can incorporate the 14-nt ssRNAs in the cell lysate. Next, those assembled RISCs were immunopurified from the cell lysate and tested for slicing activity. FLAG-AGO2 cleaved RNAs very well when the lysate was incubated with the siRNA-like duplex of miR-20a (23ds of FIG. 4) (FIG. 9B). In contrast, FLAG-AGO3 became a very competent slicer when the 14-nt single-stranded miR-20a (14ss of FIG. 4) was added to the lysate (FIG. 9C) To confirm that the observed RNA cleavage was due to the catalytic activity of AGO3, the experiment was repeated using a catalytically dead mutant, FLAG-AGO3 (E638A) (FIG. 9A₁ (Park 2017). This mutant loaded both 14-nt single-stranded miR-20a and 23-nt siRNA-like duplex of miR-20a to form the mature RISCsbut did not show slicing activity at all (FIG. 9D), proving that the catalytic center of AGO3 is essential for the cityRNA-dependent target cleavage. Lastly, it was tested whether AGO3 incorporates 14-nt single-stranded RNAs within the cell to assemble a functional slicer. The 14-nt single-stranded miR-20a was modified, according to a previous report (Lima 2012), to make it stable during and after transfection (14md of FIG. 4). When programmed with the 14-nt modified guide, the recombinant AGO3 (FIG. 2) showed a slightly higher target cleavage than with the unmodified form (FIG. 9E), indicating that the modified guide retained the ability to catalytically activate AGO3 Then, HEK293T cells were co-transfected with a plasmid encoding FLAG-AGO2 or FLAG-AGO3 and either the unmodified 14-nt miR-20a, the modified 14-nt miR-20a, or the 23-nt siRNA-like duplex of miR-20a (FIG. 9F-G). Immunopurified FLAG-AGO2 cleaved RNA very well with transfection of the 23-nt siRNA duplex (FIG. 9H). In contrast, FLAG-AGO3 cleaved the target RNA only when the modified 14-nt guide was co-transfected (FIG. 9I). Taken together, these results demonstrate that AGO3 and 14-nt ssRNAs assemble into an active slicer in vivo.

In summary, AGO2 and AGO3 have distinct guide lengths optimized for their activation. AGO2 cleaves any RNAs including a sequence fully complementary to the guide RNA, which means that any guide RNAs can activate AGO2. This is not the case for the AGO3 activation. Only specific tyRNAs can serve as cityRNAs due to their unique sequences. These multiple requirements extremely limit the opportunities for catalytically activating AGO3. It appears that AGO2 is the primary slicer under normal conditions where most of the AGO-associated miRNAs are intact, but the role is replaced by AGO3 in special conditions where 14˜15-nt guide RNAs are abundant. This model suggests that AGO3 activation needs to be strictly controlled in the cell. Otherwise, AGO3 would cleave many RNAs because 14-nt cityRNAs are about 4,000˜32,000 times more likely than 20˜23-nt guide RNAs to find a fully complementary sequence. Since AGO3 has retained the catalytic center throughout its molecular evolution, the cityRNA-dependent slicing activity could have a conserved role in or beyond RNA interference when all the requirements are met.

Materials and Methods

### Cloning, Expression, and Purification of AGO Proteins.

The genes of AGO1, AGO2, AGO3, AGO4, and FLAG-AGO3 were cloned in pFB-HTB (Invitrogen). Their recombinant proteins were purified from the insect cells as previously reported (Park 2017; Park 2019).

### In Vitro Cleavage Assay.

1 μM AGO proteins were incubated with 100 nM 5′ phosphorylated synthetic single-stranded guide RNAs for RISC assembly in 1× Reaction Buffer (25 mM HEPES-KOH, pH 7.5, 5 mM MgCl2, 50 mM KC, 5 mM DTT, 0.2 mM EDTA, 0.05 mg/mL BSA (Ambion), and 5 U/μL RiboLock RNase Inhibitor (Thermo Scientific)). 5′ cap-labeled target RNAs were added in the reaction for the target cleavage. The reaction was directly quenched with 2× urea quench dye (7 M urea, 1 mM EDTA, 0.05% (w/v) xylene cyanol, 0.05% (w/v) bromophenol blue, 10% (v/v) phenol). The cleavage products were resolved on a 7 M urea 16% (w/v) polyacrylamide gel.

### Filter Binding Assay.

1 μM AGO proteins were incubated with 0.1, 1, 5, 10, 20, 50, 100, 200 nM 5′ 32P labeled synthetic single-stranded miR-20a (p23ss) in 1× Reaction Buffer (25 mM HEPES-KOH pH 7.5, 5 mM MgCl2, 50 mM KCl, 5 mM DTT, 0.2 mM EDTA, 0.05 mg/mL BSA (Ambion), 5 U/μL RiboLock RNase Inhibitor (Thermo Scientific)) for 1 hour at 37° C. for RISC assembly. The RISC samples were spotted to Hybond ECL nitrocellulose membranes (GE Healthcare). The membranes were washed 10 times with 100 μL with 1× Binding Buffer (25 mM HEPES-KOH pH 7.5, 10 mM MgCl2, 3 mM DTT, and 125 mM NaCl), and then the dried membranes were analyzed by phosphorimager.

### Cloning, Expression, and Purification of the Isolated AGO-PAZ Domains.

The genes of isolated AGO2-PAZ (Ala227-Arg351) and AGO3-PAZ (Ala228-Arg352) domains were cloned into a sumo-fused pRSFDuet™-1 vector (2) and overexpressed in BL21(DE3) E. coli cells. The cells were homogenized in Lysis Buffer (10 mM phosphate buffer pH 7.3, 500 mM NaCl, 10 mM β-mercaptoethanol, 20 mM Imidazole, 5% Glycerol, 100 mM PMSF) and centrifuged for 50 min. The supernatant was loaded onto 5 mL HisTrap HP column (GE Healthcare) equilibrated with Buffer A1 (10 mM phosphate buffer pH 7.3, 500 mM NaCl, 10 mM β-mercaptoethanol, 20 mM Imidazole, 5% Glycerol). The column was washed with Buffer A1, followed by elution of the protein over a linear gradient to 100% of Buffer B1 (10 mM phosphate buffer pH 7.3, 500 mM NaCl, 10 mM β-mercaptoethanol, 1.5 M Imidazole, 5% Glycerol). The eluted samples were dialyzed against Buffer C1 (10 mM phosphate buffer pH 7.3, 500 mM NaCl, 10 mM, β-mercaptoethanol, 5% Glycerol) with ULP1 for overnight. The dialyzed sample was loaded onto another 5 mL HisTrap HP column (GE Healthcare) equilibrated with Buffer C1 to remove the cleaved SUMO-tag. The flow-through samples were dialyzed against Buffer D1 (10 mM Tris-HCl pH 7.5, 100 mM KC, 10 mM β-mercaptoethanol) for overnight. After concentration by ultrafiltration, the proteins were loaded onto HiLoad 16/600 Superdex 75 column (GE Healthcare) equilibrated with Buffer E1 (100 mM KCl, 10 mM Tris-HCl pH 7.5, 10 mM DTT). The purified protein was concentrated by ultrafiltration, flash-frozen in liquid nitrogen, and stored at −80° C.

### RISC Assembly Assay

10 μg pCAGEN vector encoding FLAG-AGO was transfected into HEK293T cells, and after 48 hours, the cells were harvested. Based on the western blot analysis, 50 pmol AGOs were incubated with 5 pmol 5′ end-labeled 14-nt single-strand miR-20a (p14ss in FIG. 4) or 23-nt siRNA-like duplex of miR-20a (p23ds) for RISC assembly. The RISCs were immunoprecipitated by 50 μL anti-FLAG M2 beads (Sigma-Aldrich) for 2 hours at room temperature. The immunoprecipitated RISCs were washed 8 times with Wash Buffer (300 mM NaCl, 50 mM Tris-HCl pH 7.5, 5 mM MgCl₂, and 0.05% NP-40) and 2 times with Cleavage Buffer (25 mM HEPES-KOH pH 7.5, 50 mM KCl, 5 mM MgCl_{2,} 5 mM DTT, and 2 mM EDTA). The AGO-bound RNAs were extracted with 200 μL phenol and followed by ethanol precipitated overnight. The extracted RNAs were resolved on a 10% native gel to separate the single-stranded guide RNA from the duplex (Bartel 2018).

### In Vitro Cleavage Assay Using FLAG-AGO Programmed in the Cell Lysate.

10 μg of pCAGEN vector encoding FLAG-AGO2, FLAG-AGO3, or FLAG-AGO3 (E638A) was transfected into HEK293T cells. The amount of FLAG-AGO proteins in the cell lysate was normalized based on the western blot result. AGO was quantified by using a standard curve generated with known amounts of recombinant FLAG-AGO3 (2). The lysates were incubated with 14-nt single-stranded miR-20a (14ss) or 23-nt siRNA-like duplex of miR-20a (23ds in FIG. 4) followed by immunopurification with anti-FLAG M2 beads. The beads were washed 10 times with Wash Buffer, and then the cap-labeled 60-nt target RNAs were added for target cleavage reaction. The reactions were quenched with 2× urea quench dye and resolved on a 16% polyacrylamide gel.

Validation of Modified 14-Nt miR-20a by In Vitro Cleavage Assay

1 μM recombinant AGO3 was incubated with a 14-nt unmodified single-stranded miR-20a (14ss), a 14-nt modified single-stranded miR-20a (14md in FIG. 4), or a 23-nt single-stranded miR-20a (23ss in FIG. 4) for 1 hour at 37° C., followed by incubation with the cap-labeled 60-nt target RNAs to start the cleavage reaction. The reaction was stopped with 2× urea quenching dye and resolved on a 7 M urea 16% polyacrylamide gel.

In Vitro Cleavage Assay Using FLAG-AGO Programmed within the Cell

10 μg of pCAGEN vector encoding FLAG-AGO2, FLAG-AGO3, or FLAG-AGO3 (E638A) was transfected into HEK293T cells. After 24 hours, 14-nt unmodified single-stranded miR-20a (14ss in FIG. 4), 14-nt modified single-stranded miR-20a (14md in FIG. 4), or 23-nt siRNA-like duplex of miR-20a (23ds in FIG. 4) was transfected. 24 hours later, the cells were harvested and lysed by sonication. The amount of FLAG-AGO proteins in the cell lysate was normalized based on the western blot result. AGO was quantified by using a standard curve generated with known amounts of recombinant FLAG-AGO3 (Bartel 2018). The overexpressed FLAG-AGOs were immunoprecipitated with 50 μL anti-FLAG M2 beads. After the beads were washed with Wash Buffer, the cap-labeled 60-nt target RNAs were added for cleavage reaction.

### Example 2: Requirements of cityRNAs and Targets Cleaved by AGO3

Determine the Requirements of cityRNA and AGO3 for Catalytic Activation.

It was found that the 14-nt miR-20a and let-7a activate AGO3 for RNA cleavage. The two cityRNAs share A, G, and U at g3 (guide nucleotide position 3), g5, and g6, respectively, none of which is found in the 14-nt miR-16 or miR-19b which do not activate AGO3. The three nucleotides are replaced in the 14-nt miR-20a and let-7a while incorporated into the 14-nt miR-16 and miR-1 Ob to validate their effect on target cleavage. A previous study revealed that AGO3 possesses unique local structures (Park 2004). To examine the involvement of these local structures in recognition of cityRNAs, AGO3 mutants are made lacking either of the unique motifs and they are tested for in vitro cityRNA-directed RNA cleavage. The crystal structures of AGO3 in complex with the 14-nt miR-20a or let-7a are determined to understand how AGO3 recognizes cityRNAs.

Determine the Requirements of Target RNAs for Cleavage by cityRNA-Loaded AGO3.

The mechanism of target cleavage by AGO2 is reported to be that targets are paired sequentially with the seed region (g2-g8) of the bound guide RNA (19˜23 nt), the 3′ supplementary region (g13-g16), and the central region (g9-g12) in this order, as a prerequisite for cleavage (Bartel 2018; Sheu 2019). In contrast, 14-nt cityRNAs cannot form a stable duplex in its 3′ supplementary region, suggesting that AGO3 takes a different activation manner. To determine the sequence complementarity between cityRNA and target RNAs required for target cleavage, a single nucleotide mismatch is incorporated at every position of the 14-nt miR-20a and let-7a. After loading with either of the cityRNAs, AGO3 is tested for in vitro slicing activity as reported (Dayeh 2018). A previous study revealed that AGO3 loaded with 23-nt miR-20a requires both 5′ and 3′ regions flanking the target site to cleave the RNAs (Park 2017). Target RNA variants are made and it is determined the minimum length of target RNA required for cityRNA-directed RNA cleavage, The ternary complex crystal structures of AGO3 is determined with the 14-nt miR-20a or let-7a and their target RNA. The complex structures of AGO3 with the 14-nt miR-16 or miR-19b and their target also are determined to understand why they do not activate AGO3.

### Results

AGO3 showed a noticeable slicing activity when loaded with a 14 nt miR-20a. In 2004, two groups independently tested four human AGOs programmed with several mi RNAs for in vitro target cleavage and showed that only AGO2 cleaved RNAs (Liu 2004; Park 2017). Since then, it has been thought that the rest of human paralogs, AGO1, AGO3, and AGO4, serve as slicer-independent AGOs. However, AGO3 shares exactly the same catalytic DEDH tetrad with AGO2, which raised a question of why AGO3 has conserved the catalytic residues throughout its molecular evolution. This question was partially answered by a recent study that only specific miRNAs such as miR-20a, but neither let-7a, miR-16, nor miR-19b, catalytically activated AGO3 (Park 2017). The observed activity was, however, much lower than that of AGO2. Meanwhile, it was observed that a budding yeast, Kluyveromyces polysporus, Ago1 retained about 40 percent of the slicing activity with even a 14-nt guide RNA (Dayeh 2018). This result prompted a test whether such a short guide RNA can activate AGO3. To validate the idea, recombinant AGO2 and AGO3 proteins were expressed in and purified from insect cells as reported previously (Park 2017). Then, AGO2 and AGO3 were programmed with 8-, 10-, 12-, 13-, 14-, 15-, 16-, or 23-nt miR-20a, followed by incubation with a 60-nt 5′ cap-labeled target that includes a sequence (t1-t23: target nucleotide positions 1-23) complementary to the guide nucleotide positions 1-23 (g1-g23). AGO2 reduced the slicing activity as it loaded a shorter guide. In contrast, AGO3 showed the highest cleavage percentage when loaded with the 14-nt miR-20a. Notably, the slicing activity of AGO3 with the 14-nt miR20a was about 20 times as high as that with the 23-nt miR-20a. To exclude a possibility that the observed differences in target cleavage with 8˜23-nt guide RNAs were attributed to their loading efficiencies, filter-binding assays were performed where AGO2 and AGO3 were incubated with the 10-, 14-, or 23-nt miR-20a whose 5′ end is 32P labeled. Both AGOs showed similar loading efficiency regardless of the guide length. Collectively, the results demonstrate that the 14-nt miR-20a catalytically activates AGO3. Given that all the previous cleavage assays of human AGOs were performed with a 21˜23-nt guide RNA, the results can explain why nobody has noticed the substantial slicing activity of AGO3.

### AGO3 Became a Competitive Slicer Like AGO2.

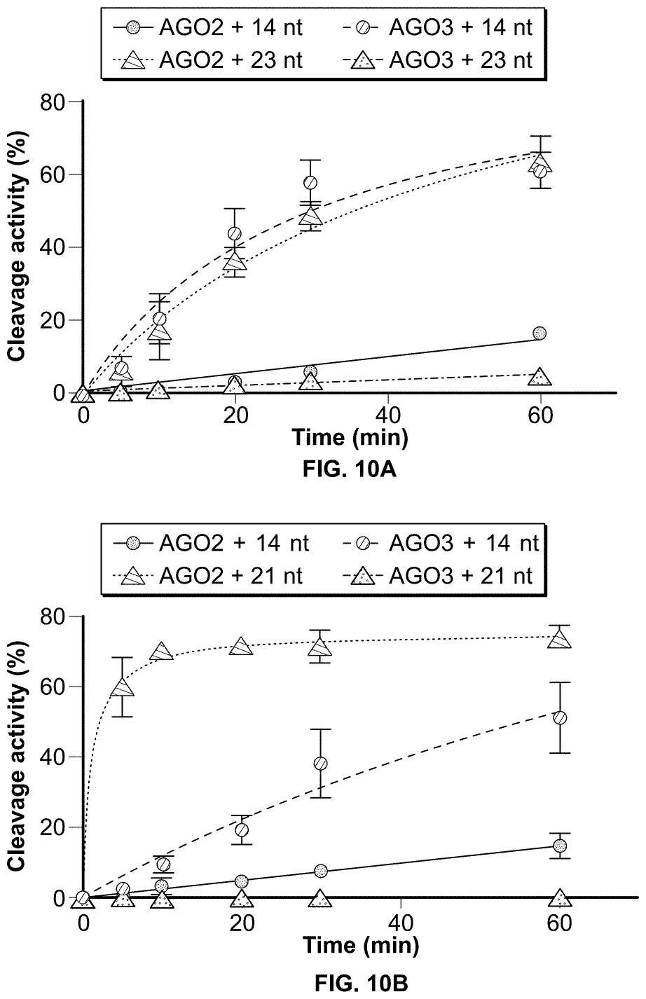

Next, a time-course assay was performed by sampling an aliquot of the reaction at 20, 40, and 60 min. As a result, AGO3 loaded with the 14-nt miR-20a showed a slicing activity similar to that of AGO2 with the 23-nt miR-20a (FIG. 10a). These results suggest that AGO2 and AGO3 have different optimum lengths of guide RNA for target cleavage. To validate the idea, AGO2 and AGO3 were programmed with a 21- or 14-nt let-7a, the latter of which corresponds to the g1-g14 of the 21-nt let-7a. Then, their RISCs were incubated with a 60-nt 5′ cap-labeled target that includes a fully complementary sequence. As a result, the 14-nt let-7a conferred decent slicing activity on AGO3 whereas it converted AGO2 to a much weaker slicer compared with the 21-nt let-7a (FIG. 10b). In both miR-20a and let-7a, AGO3 loaded with the 14-nt guide showed higher slicing activity than AGO2 with their intact form (i.e., the 23-nt miR-20a and the 21-nt let-7a) (FIGS. 10a-b). These results show that intact and short miRNAs adequately activate AGO2 and AGO3, respectively.

AGO3 Loaded with the 14-nt miR-20a Cleaved Target RNAs as does AGO2 with the 23-nt miR-20a.

It is well known that AGOs count the cleavage site from the 5′ end of the bound guide strand and cleave the target strand between t10 and t11. Although AGO3 cleaved target RNA when loaded with the 14-nt mirR-20a, the cleavage site remained unclear. When loaded with the 14- or 23-nt miR-20a, AGO2 and AGO3 generated cleavage products of the same size, indicating that both AGOs cleave target RNAs at the same position, regardless of the guide length.

The 14-nt miR-20a Catalytically Activated Neither AGO1 Nor AGO4.

It was shown that changing the length of guide RNA from 23 nt to 14 nt increased the slicing activity of AGO3 by about 20-fold. Although previously determined structures revealed that AGO1 and AGO4 have a pseudo catalytic tetrad, DEDR, and DEGR, respectively (Nakanishi 2013; Park 2019; Faehnle 2013), it could not be excluded that the 14-nt miR-20a can convert AGO and AGO4 to a decent slicer. To test the idea, four human AGOs were programmed with the 14- or 23-nt miR-20a, followed by addition of the 60-nt 5′ cap-labeled target RNA. AGO2 and AGO3, but not AGO1 or AGO4, cleaved RNAs. Therefore, AGO2 and AGO3 are the only slicers.

Not all 14-nt miRNA Derivatives can Catalytically Activate AGO3.

AGO2 cleaved target RNAs if the loaded intact miRNA is perfectly paired with target strands, regardless of the guide sequence. In contrast, AGO3 showed a substantial slicing activity when loaded with the 14-nt miR-20a and let-7a but with neither a 14-nt miR-16 nor miR-19b, both of which correspond to the g1-g14 of their intact miRNA. The result indicates that 14-nt derivatives of only specific miRNAs can convert AGO3 to a slicer. This is another reason why it has been difficult to discover the slicing activity of AGO3. These results indicate that AGO2 and AGO3 have different activation mechanisms although both cleave target RNAs at the same position.

Hereafter, ˜18-nt miRNA derivatives are named tiny RNAs (tyRNA) to distinguish from their intact miRNAs (19˜23-nt). In addition, tyRNAs capable of catalytically activating AGO3, such as the 14-nt miR-20a and let-7a, are referred to as cleavage-inducing tyRNAs (cityRNAs). On the other hands, tyRNAs incapable of activating AGO3, such as the 14-nt miR-16 and miR-19b, are called non-cityRNAs.

### AGO3-RISC Possesses Several Unique Local Structures.

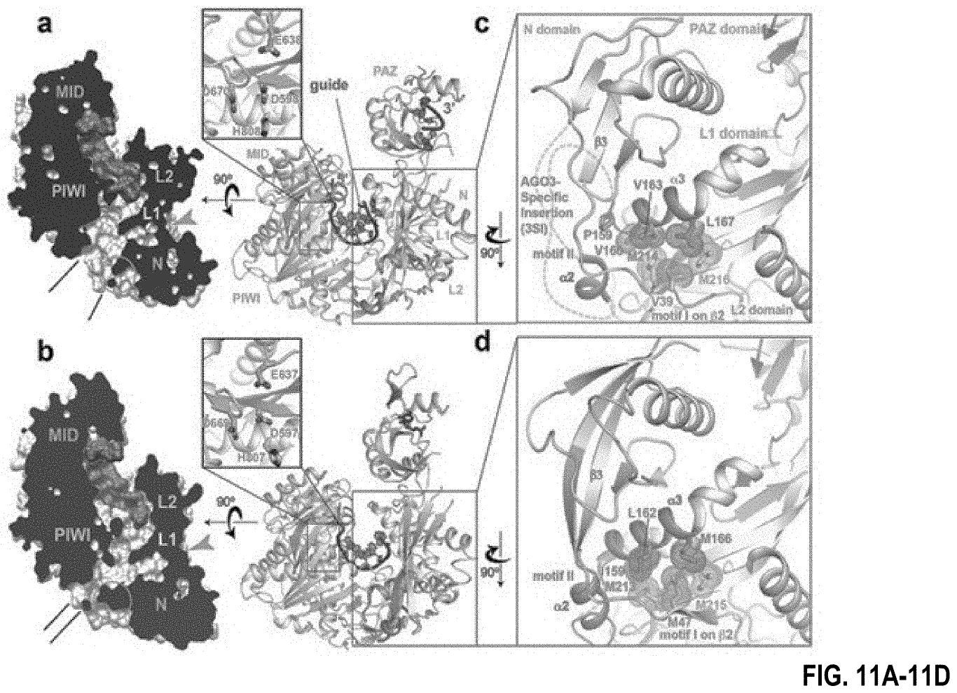

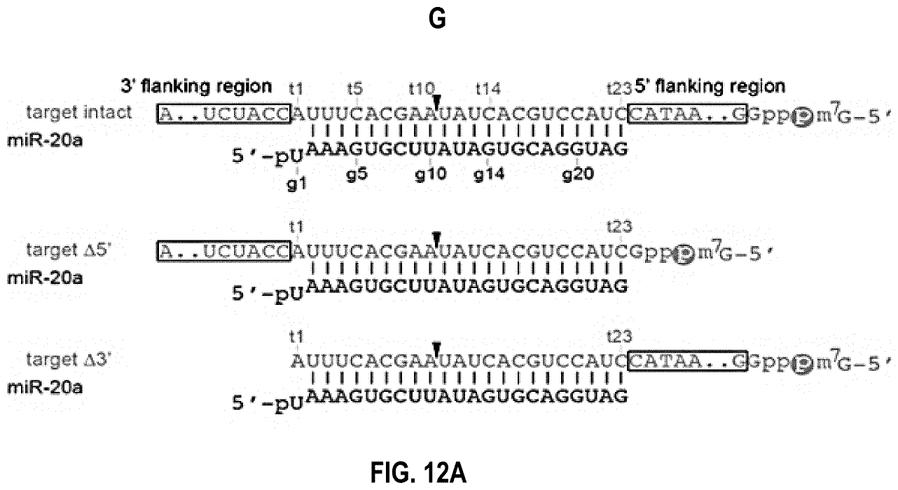

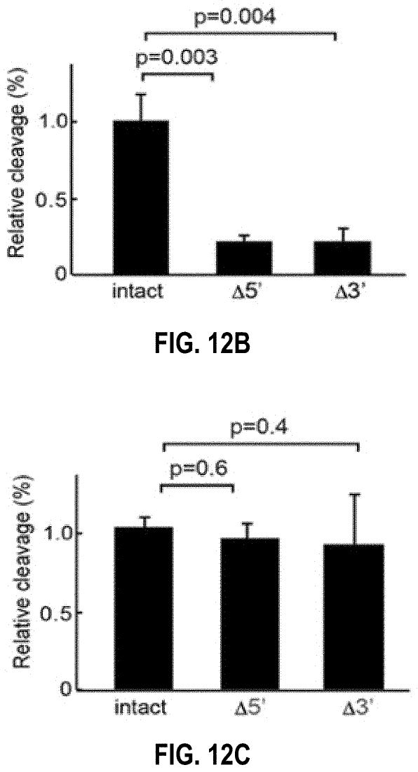

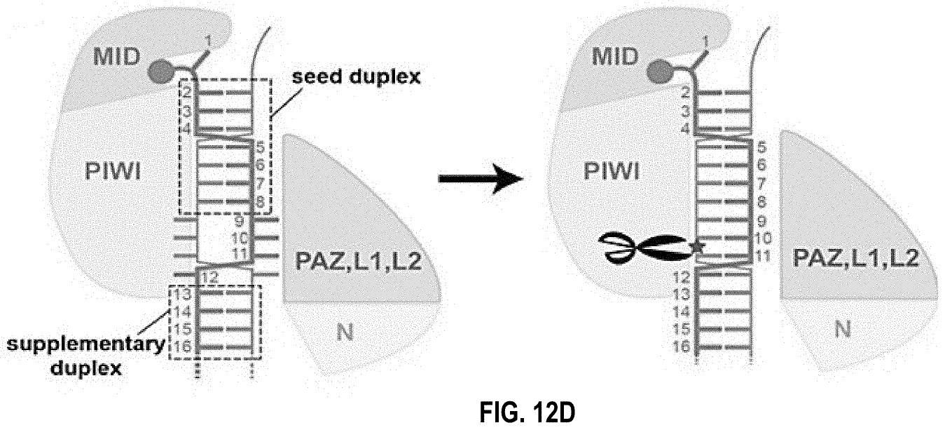

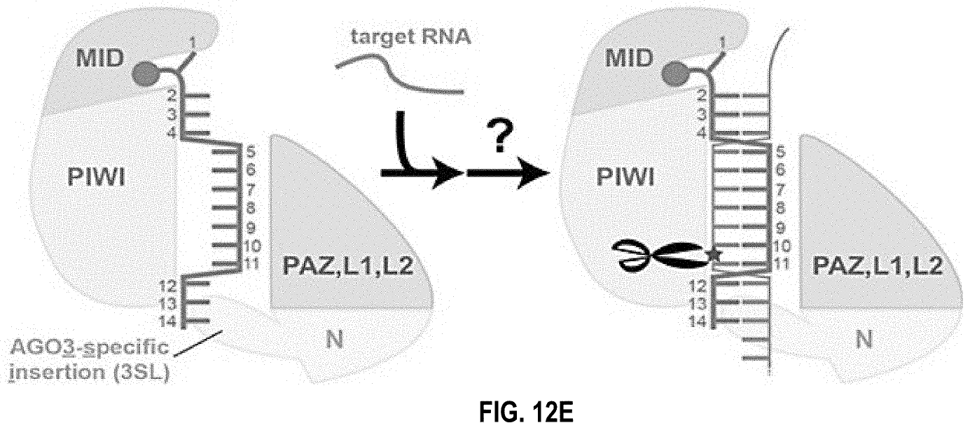

The first crystal structure of human AGO3 in complex with guide RNA (AGO3-RISC) was recently established (Park 2017). The structure revealed that AGO3 completed the catalytic DEDH tetrad like AGO2 (FIG. 11a-b), indicating that AGO3-RISC is ready to cleave target RNAs. However, AGO3-RISC has the following three structural features that are different from AGO2. First, the nucleic acid binding channel forms a different shape, due to its unique AGO3-Specific Insertion (3SI) (dotted parts in FIG. 11c). The structure suggests that despite its poor electron density, the 3S1 interacts with the 3′ half of guide strand (g13˜) and the target (t13˜) (also see FIG. 12e). Second, the AGO3 N and L1 domains are packed loosely because short, non-polar residues form some empty space in their interface (FIGS. 11a and c). In contrast, the AGO2 N and L1 domains are packed tightly because the corresponding non-polar residues have longer side chains and thus fill the space (FIGS. 11b and d). Third, the AGO3 N domain locates 13 unique residues near the 3SI. Given that AGO3 is activated efficiently by only cityRNAs, it is plausible that the above-mentioned structural features enable AGO3 to recognize the length, or specific nucleotides of cityRNAs, or both.

### Mechanisms of Target RNA Cleavage are Different Between AGO2 and AGO3.

A recent study revealed that AGO3 loaded with the 23-nt miR-20a drastically reduced the slicing activity when the target site lacks either 5′ or 3′ flanking region (FIG. 12a-b) (Park 2017). This result suggests that both flanking regions are essential for sufficient target cleavage of AGO3 when loaded with intact miRNA. In contrast, AGO2 loaded with the 23-nt miR-20a cleaved the target RNAs despite the lack of 5′ or 3′ flanking region (FIG. 12c). These observations suggest that AGO2 and AGO3 recognize and cleave target RNAs in different manners when loaded with an intact miRNA (19˜23 nt). Recently, the following model of target cleavage by AGO2 was proposed based on the accumulated biochemical and structural data (Bartel 2018). First, target strands are paired with the seed region (g2-g8) of the AGO2-bound guide RNA and subsequently with the supplementary region (g13˜) (FIG. 12d left). The target is not yet paired with the central region of the guide (g9-g12). Second, only when the resultant seed duplex and the supplementary duplex are fully complementary, the two duplexes are twisted so that the target can form base pairings with the central region (FIG. 12d right). Finally, AGO2 cleaves the target between t10 and t11 if the target is fully paired with the central region. The model was supported by the very recent crystal structure of the ternary complex of AGO2 with a 21-nt guide RNA and its complementary target RNA (Sheu 2019). An AGO2 catalytic mutant was employed in this study to avoid target cleavage during crystallization because a fully complementary target strand was used. Nevertheless, the structure showed that the seed and supplementary duplexes were formed whereas the central region was not paired, which is consistent with the proposed model (FIG. 12d left). However, the AGO2 activation model does not seem to be applicable to cityRNA-loaded AGO3 because 14-nt cityRNAs have only two nucleotides (i.e., g13-g14), which is too short to form a stable supplementary duplex (FIG. 12e). These results prompted the hypothesis that AGO3 must take a different mechanism to recognize and cleave target RNAs.

### RNA Sequencing of AGO3-Bound RNAs.

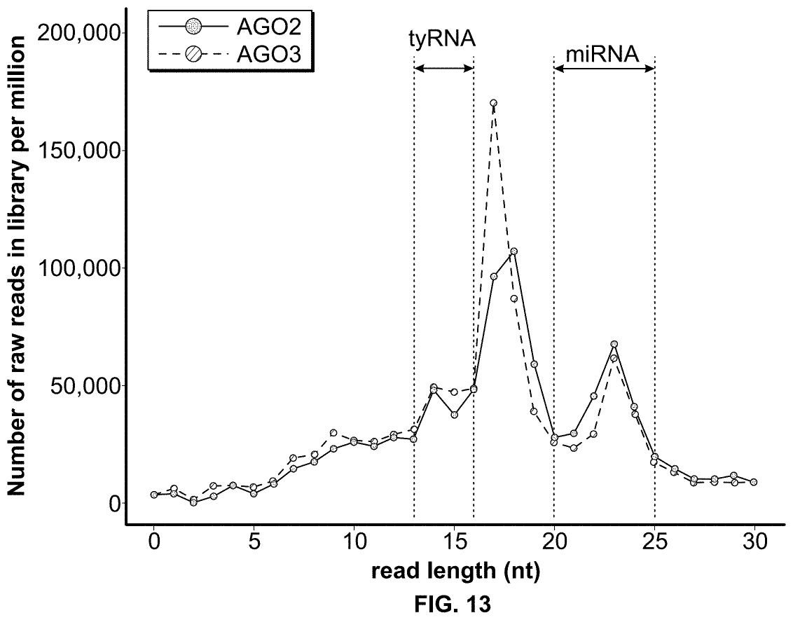

Most of the previous studies about miRNAs using next generation RNA sequencing (RNAseq) have focused on reads mapped on mature miRNAs within a range of 19˜23 nt. Any RNAs shorter than 19 nt were usually excluded from the analyses. Therefore, little is known about AGO-bound shorter RNAs including tyRNAs whose size is ˜18 nt. FLAG-AGO2 or FLAG-AGO3 was expressed in HEK293T cells and immunopurified them with anti-FLAG beads, followed by RNA extraction. Notably, the custom RNAseq library preparation approach that was used captures RNAs irrespective of their lengths, and is particularly advantageous to identify RNAs as short as 10 nt. As a result, the length distribution of the AGO-bound RNAs showed three populations with their peaks at 14, 18, and 23 nt (FIG. 13). The findings demonstrated that both AGO2 and AGO3 bind to tyRNAs in the cell. Given that AGO3 has retained the catalytic tetrad throughout its molecular evolution, it is hypothesized that AGO3 cleavage with cityRNA must play important roles although such physiological functions have not been identified yet.

### Experimental Design

Determine Specific Nucleotides within cityRNAs Essential for the Catalytic Activation of AGO3.

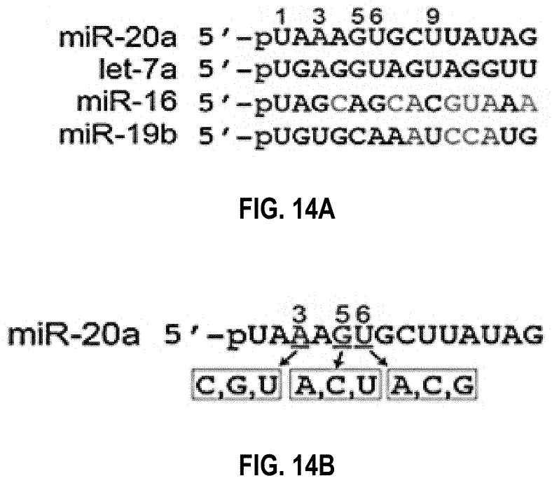

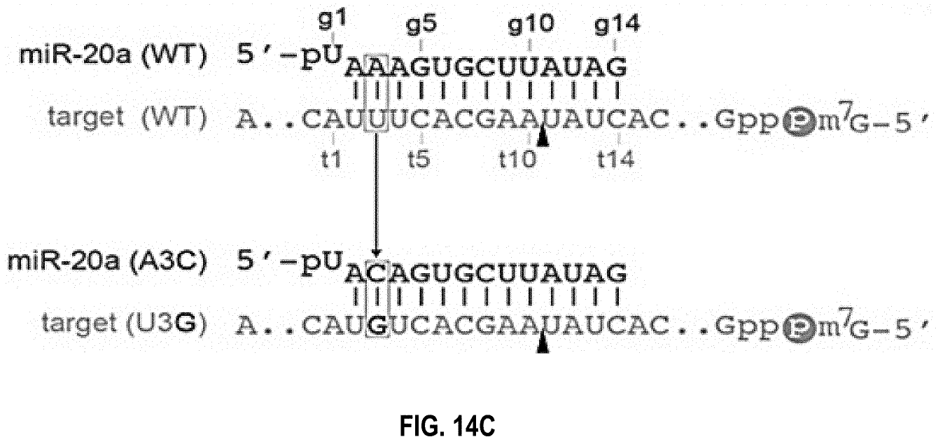

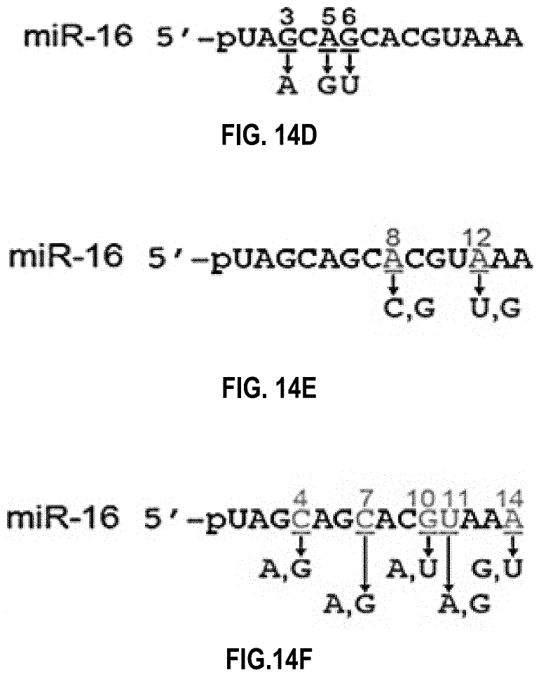

A preliminary study revealed that the 14-nt tyRNAs of miR-20a and let-7a, but not of miR-16 or miR-19b, converted AGO3 to a strong slicer. The tyRNAs of miR-20a and let-7a share five nucleotides, U1, A3, G5, U6, and U9 (FIG. 1ra). Among these, only A3, G5, and U6 are unique to miR-20a and let-7a but not to miR-16 or miR-19b (FIG. 14a), suggesting that these three nucleotides are possible determinants for cityRNA-directed RNA cleavage. To test this idea, each of the three nucleotides on the 14-nt miR-20a will be replaced with a different one (FIG. 14b). Meanwhile, the pairing nucleotide on the target RNA also are replaced so that the cityRNA variant and its target maintain their complementarity (FIG. 14c). Recombinant AGO2 and AGO3 proteins are expressed in and purified from insect cells. After loading with either of the cityRNA mutants, the purified AGO2 and AGO3 are incubated with the 60-nt 5′ cap-labeled target RNA mutant. The cleavage product is separated from the intact target on a 7 M urea denaturing 16% polyacrylamide gel. The gel is dried and analyzed by phosphorimaging. The band intensities of the intact target and the cleavage product are quantified using ImageQuant. The cleavage percentages are compared between the cityRNA wild-type and its variants. The same assay is performed using the 14-nt let-7a. Once nucleotides are identified as serving as positive determinants for the cityRNA-directed RNA cleavage, those nucleotides are incorporated into non-cityRNAs, such as the 14-nt miR-16 and miR-19b, to validate their effect (FIG. 14d).

Determine AGO3-Specific Motifs Required for cityRNA-Directed RNA Cleavage.

Human AGOs share amino acid sequence identities of about 80% (Sasaki 2003), suggesting that they must be folded into quite similar protein structures. The crystal structure of four human AGOs revealed that despite the similarity of their overall structures, each AGO possesses unique local structures (Park 2017). The AGO3 structure showed that the N domain possessed an AGO3-specific insertion (3SI), which results in a different shape of the nucleic acid-binding channel from that of AGO2 (FIGS. 11a-b). The AGO3 N domain locates many unique amino acid residues on the surface that are not seen in the other AGO paralogs. Furthermore, the AGO3 N domain and L1 domain do not interact with each other tightly due to their loose packing (FIGS. 11c-d).

Based on the structural differences between AGO2 and AGO3 (Park 2017), recombinant AGO3 mutants are systemically designed in which either of the specific local structures is replaced with the corresponding part of AGO2. Those mutants are expressed in and purified from insect cells. Synthetic guide RNAs and 5′ cap labeled target RNAs are prepared. After being programmed with the 14-nt miR-20a, the purified AGO3 mutants are incubated with the 60-nt 5′ cap-labeled target RNA. The reactions are resolved on a 7 M urea denaturing 16% polyacrylamide gel to quantify the cleavage percentages. To examine the significance of the AGO3-specific local structures for other tyRNAs, the same experiment is performed using 14-nt let-7a, miR-16, and miR-19b. Last, the same experiments are performed with their 23-nt guides to validate whether the observed results are tyRNA-dependent.

Determine the Binary Structure of AGO3 in Complex with a cityRNA.

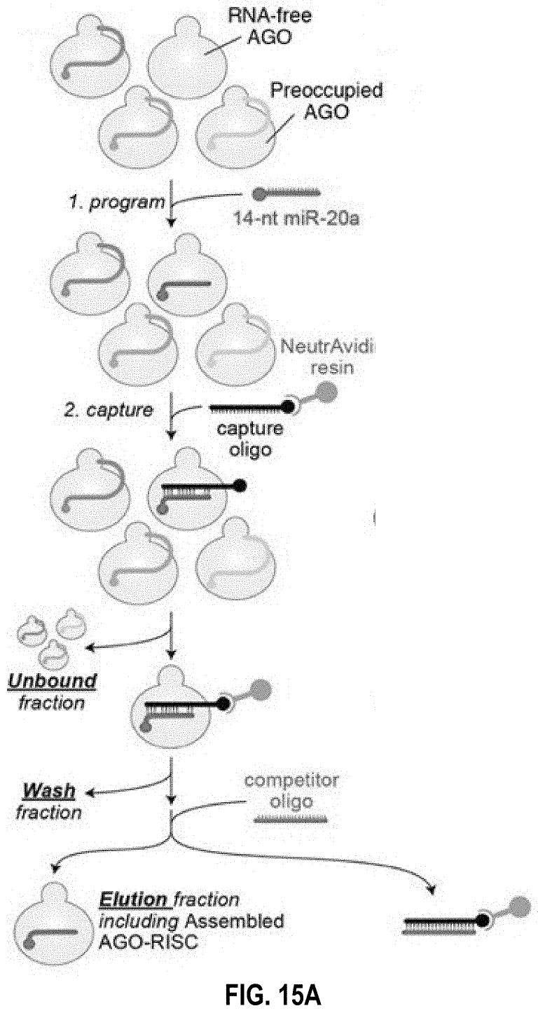

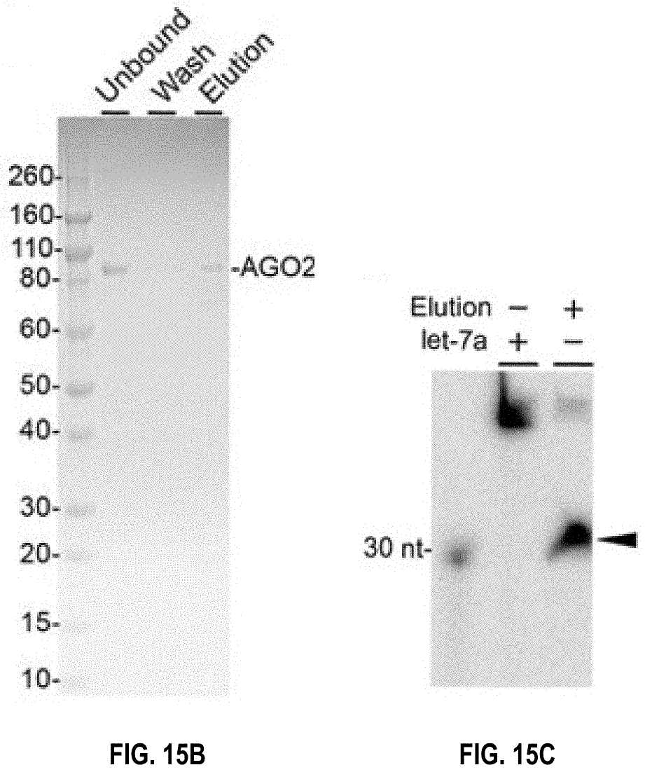

A recombinant AGO3 protein is expressed in insect cells and purify a homogeneous AGO3-RISC that is programmed with the 14-nt miR-20a or let-7a. To this end, a procedure that is modified based on the previously reported Arpón method is used (FIG. 15a)(Schirle 2014; Flores 2012). A homogeneous AGO2 loaded with the 21-nt let-7a was purified that showed very high cleavage activity (FIGS. 15b-c). The same method is employed to purify the homogeneous AGO3 loaded with the specific cityRNA, followed by initial screening of crystallization conditions using a robot, Mosquito (TTP), as previously performed (Park 2017). Once obtaining preliminary crystals, their diffraction is tested in a X-ray diffractometer. When their diffraction quality is good, we the crystallization conditions are optimized further. Obtained best crystals are shipped to NE-CAT at Advanced Photon Source (Chicago), and data sets are collected remotely. The binary structure of AGO3 with the 14-nt cityRNA is determined by molecular replacement using a previously determined AGO3 structure (PDB: 5VM9) as a search model.

### Experimental Design

Determine the Extent of Complementarity Between cityRNA and Target RNAs Required for Target Cleavage.

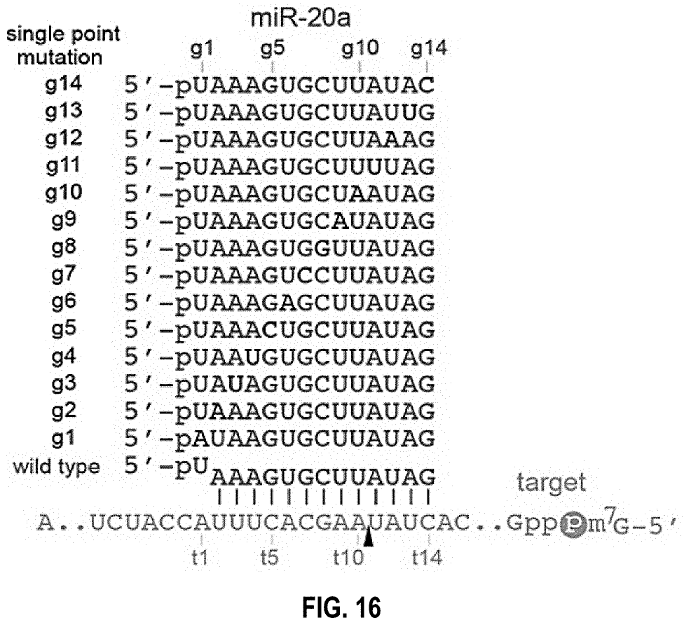

To evaluate whether a tyRNA works as a cityRNA (i.e., whether the tyRNA activates AGO3 for RNA cleavage), target RNAs were used that include a sequence perfectly complementary to the corresponding miRNA (e.g., when AGO3 is programmed with the 14-nt miR-20a, the target RNA includes a sequence complementary to the 23-nt miR-20a). It remains unclear whether RNA cleavage by AGO3 is tolerant to mismatches between the cityRNA and target RNAs, and, if so, whether the position of the mismatch causes different effects on the target cleavage. A series of synthetic RNAs of the 14-nt miR-20a and let-7a variants are used that include a single nucleotide mismatch at a different position (FIG. 16). Recombinant AGO3 protein are expressed in and purified from insect cells as described in 2.1 (Park 2017). After loading with either of the 14-nt miR-20a or let-7a variants, AGO3 is incubated with the 60-nt 5′ cap-labeled target RNA. The reaction is dissolved and analyzed as described herein. The cleavage percentages between the cityRNA wild-type and its variant are compared.

Determine the Minimum Length of Target RNAs Cleaved by cityRNA-Loaded AGO3.

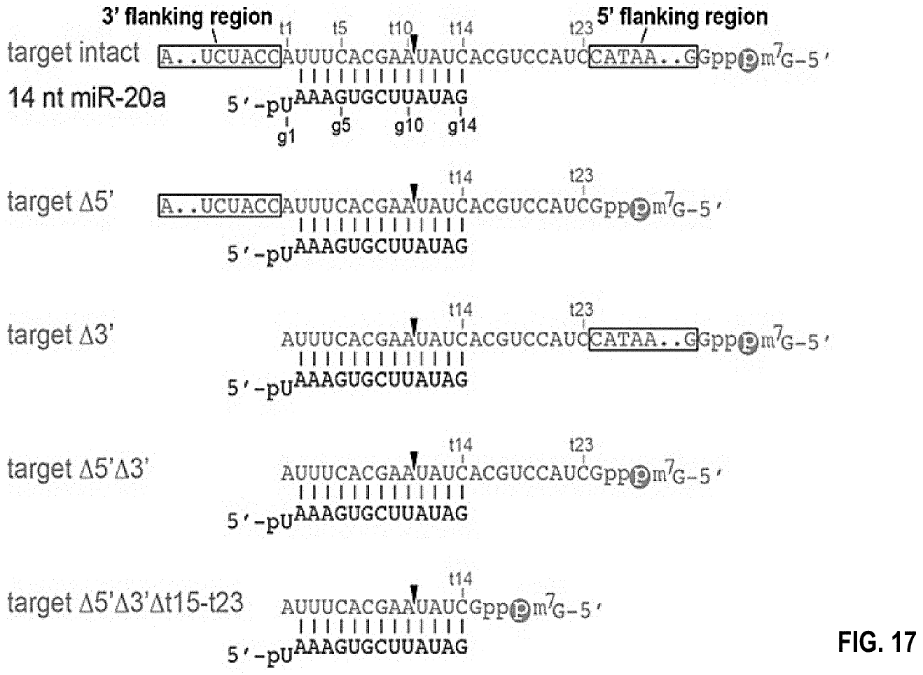

It was previously reported that when loaded with the 23-nt miR-20a, AGO2 cleaved a fully complementary target RNA that even lacks a 5′ or 3′ flanking region (FIG. 12c) (Park 2017) In contrast, AGO3 loaded with the 23-nt miR-20a did not cleave those targets, suggesting a possibility that AGO3 requires both flanking regions for RNA cleavage. After that, it was discovered that the 14-nt miR-20a and let-7a convert AGO3 to a competitive slicer. These results prompted a test whether such a cityRNA-loaded AGO3 also requires flanking regions for RNA cleavage. To this end, AGO3 is loaded with the 14-nt miR-20a, followed by incubation with the shorter target RNAs, Δ5′ and Δ3′ (FIG. 17), which are the same targets used in FIG. 12. If the cityRNA-loaded AGO3 cleaves the two targets, a shorter target RNA is made, Δ5′Δ3′, that lacks both flanking regions (FIG. 17). When the cityRNA-loaded AGO3 cleaves the target Δ5′Δ3′, an even shorter target, Δ5′Δ3′Δt15-t23, that further lacks t15-t23 is made (FIG. 17). The cleavage percentages is quantified as described herein The abovementioned assays also are performed using the 14-nt let-7a to examine whether the effect of the 5′ and 3′ flanking regions and the t15-t23 changes when AGO3 is programmed with different cityRNAs. Negative control experiments are done using non-cityRNAs such as the 14-nt miR-16 and miR-19b. In addition, the same set of the short target RNAs, Δ5′Δ3′ and Δ5′Δ3′Δt15-t23, are tested for cleavage by AGO2 loaded with the 14- or 23-nt miR-20a, to determine the minimum target length.

Determine the Ternary Complex Structure of cityRNA-Loaded AGO3 with a Target RNA.

As described above, it is believed that cityRNA-loaded AGO3 recognizes target RNAs in a different manner from that of miRNA-loaded AGO2. To elucidate the molecular mechanism, it is determined the crystal structure of AGO3 in complex with the 14-nt miR-20a and a target RNA. To avoid target cleavage during the crystallization, a catalytically inactive mutant, AGO3 (D670A) is made. The corresponding mutant AGO2 (D669A) was used for the structure determination of the target complex of miRNA-loaded AGO2 (Sheu 2019). The recombinant AGO3 (D670A) is expressed in insect cells and the Arpón method is used to purify a homogeneous AGO3 (D670A) loaded with the 14-nt miR-20a as shown in FIG. 15. The purified cityRNA-loaded AGO3 (D670A) are incubated with a target RNA that includes a sequence fully complementary to the 14-nt miR-20a. Screening and optimization of the crystallization conditions, data collection, and, structure determination are performed similarly as described above. The ternary structure also is determined with another cityRNA, 14-nt let-7a, as well as with non-cityRNAs, 14-nt miR-16 and miR-19b.

The compositions, devices, systems, and methods of the appended claims are not limited in scope by the specific compositions, devices, systems, and methods described herein, which are intended as illustrations of a few aspects of the claims. Any compositions, devices, systems, and methods that are functionally equivalent are intended to fall within the scope of the claims. Various modifications of the compositions, devices, systems, and methods in addition to those shown and described herein are intended to fall within the scope of the appended claims. Further, while only certain representative compositions, devices, systems, and method steps disclosed herein are specifically described, other combinations of the compositions, devices, systems, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

Unless defined otherwise, all technical and scientific terms used herein have the same meanings as commonly understood by one of skill in the art to which the disclosed invention belongs. Publications cited herein and the materials for which they are cited are specifically incorporated by reference.

### REFERENCES

1. Bartel D P. Metazoan MicroRNAs. Cell. 2018; 173(1):20-51 doi: 10.1016/j.cell.2018.03.006. PubMed PMID: 29570994.
2. Liu J, Carmell M A, Rivas F V, Marsden C G, Thomson J M, Song J J, Hammond S M, Joshua-Tor L, Hannon G J. Argonaute2 is the catalytic engine of mammalian RNAi. Science. 2004; 305(5689):1437-41. Epub 2004/07/29. doi: 10.1126/science.1102513. PubMed PMID: 15284456.
3. Meister C, Landthaler M, Patkaniowska A, Dorsett Y, Teng G, Tuschl T. Human Argonaute2 mediates RNA cleavage targeted by miRNAs and siRNAs. Mol Cell. 2004; 15(2):185-97. doi: 10.1016/j.molcel.2004.07.007. PubMed PMID: 15260970.
4. Park M S, Phan H D, Busch F, Hinckley S H, Brackbill J A, Wysocki V H, Nakanishi K. Human Argonaute3 has slicer activity. Nucleic Acids Res. 2017; 45(20):11867-77. doi: 10.1093/nar/gkx916. PubMed PMID: 29040713; PMCID: PMC5714244.
5. Sheu-Gruttadauria J, Xiao Y, Gebert L F, MacRae I J. Beyond the seed: structural basis for supplementary microRNA targeting by human Argonaute2. EMBO J. 2019; 38(13):e101153. Epub 2019/07/04. doi: 10.15252/embj.2018101153, PubMed PMID: 31268608.
6. Dayeh D M, Kruithoff B C, Nakanishi K. Structural and functional analyses reveal the contributions of the C- and N-lobes of Argonaute protein to selectivity of RNA target cleavage. J Biol Chem. 2018; 293(17):6308-25. Epub 2018/03/08. doi: 10.1074/jbc.RA117.001051. PubMed PMID: 29519815; PMCID: PMC5925815.
7. Zheng Z, Wang L, Pan J. Interferon-stimulated gene 20-kDa protein (ISG20) in infection and disease: Review and outlook. Intractable Rare Dis Res. 2017; 6(1):35-40. Epub 2017/03/31. doi: 10.5582/irdr.2017.01004. PubMed PMID: 28357179; PMCID: PMC5359350.
8. Gangras P, Dayeh D M, Mabin J W, Nakanishi K, Singh G. Cloning and Identification of Recombinant Argonaute-Bound Small RNAs Using Next-Generation Sequencing. Methods Mol Biol. 2018; 1680:1-28. Epub 2017/10/17. doi: 10.1007/978-1-4939-7339-2_1. PubMed PMID: 29030838.
9. Kozomara A, Birgaoanu M, Griffiths-Jones S. miRBase: from microRNA sequences to function. Nucleic Acids Res. 2019; 47(D1):D155-D62. Epub 2018/11/14. doi: 10.1093/nar/gky1141. PubMed PMID: 30423142; PMCID: PMC6323917.
10. Zhang H, Kolb F A, Jaskiewicz L, Westhof E, Filipowicz W. Single processing center models for human Dicer and bacterial RNase III. Cell. 2004; 118(1):57-68. Epub 2004/07/10. doi: 10.1016/j.cell.2004.06.017. PubMed PMID: 15242644.
11. Macrae I J, Zhou K, Li F, Repic A, Brooks A N, Cande W Z, Adams P D, Doudna J A. Structural basis for double-stranded RNA processing by Dicer. Science. 2006; 311(5758):195-8. Epub 2006/01/18. doi: 10.1126/science.1121638. PubMed PMID: 16410517.
12. MacRae I J, Zhou K, Doudna J A. Structural determinants of RNA recognition and cleavage by Dicer. Nat Struct Mol Biol. 2007; 14(10):934-40. Epub 2007/09/18. doi: 10.1038/nsmb1293. PubMed PMID: 17873886.
13. Nakanishi K. Anatomy of RISC: how do small RNAs and chaperones activate Argonaute proteins? Wiley Interdiscip Rev RNA. 2016; 7(5):637-60. Epub 2016/05/18. doi: 10.1002/wrna.1356. PubMed PMID: 27184117; PMCID: PMC5084781.
14. Meister G. Argonaute proteins: functional insights and emerging roles. Nat Rev Genet. 2013; 14(7):447-59. Epub 2013/06/05. doi: 10.1038/nrg3462. PubMed PMID: 23732335.
15. Wilson R C, Doudna J A. Molecular mechanisms of RNA interference. Annu Rev Biophys. 2013; 42:217-39. Epub 2013/05/10. doi: 10.1146/annurev-biophys-083012-130404. PubMed PMID: 23654304; PMCID: PMC5895182.
16. Jinek M, Doudna J A. A three-dimensional view of the molecular machinery of RNA interference. Nature. 2009; 457(7228):405-12. Epub 2009/01/23. doi: 10.1038/nature07755. PubMed PMID: 19158786.
17. Kuscu C, Kumar P, Kiran M, Su Z, Malik A, Dutta A. tRNA fragments (tRFs) guide Ago to regulate gene expression post-transcriptionally in a Dicer-independent manner. RNA. 2018; 24(8):1093-105. Epub 2018/05/31. doi: 10.1261/rna.066126.118. PubMed PMID: 29844106; PMCID: PMC6049499.
18. Baldrich P, Rutter B D, Karimi H Z, Podicheti R, Meyers B C, Innes R W. Plant Extracellular Vesicles Contain Diverse Small RNA Species and Are Enriched in 10- to 17-Nucleotide “Tiny” RNAs. Plant Cell. 2019; 31(2):315-24. Epub 2019/02/02. doi: 10.1105/tpc.18.00872. PubMed PMID: 30705133; PMCID: PMC6447009.
19. Wittrup A, Lieberman J. Knocking down disease: a progress report on siRNA therapeutics. Nat Rev Genet. 2015; 16(9):543-52. Epub 2015/08/19. doi: 10.1038/nrg3978. PubMed PMID: 26281785; PMCID: PMC4756474.
20. Kannan R M. Toward new design principles for superior gene silencing. Proc Natl Acad Sci USA. 2018; 115(13):3200-1. Epub 2018/03/20. doi: 10.1073/pnas.1801554115. PubMed PMID: 29549152; PMCID: PMC5879714.
21. Cheloufi S, Dos Santos C O, Chong M M, Hannon G J. A dicer-independent miRNA biogenesis pathway that requires Ago catalysis. Nature. 2010; 465(7298):584-9. Epub 2010/04/29. doi: 10.1038/nature09092. PubMed PMID: 20424607: PMCID: PMC2995450.
22. Tokita M J, Chow P M, Mirzaa C, Dikow N, Maas B, Isidor B, Le Caignec C, Penney L S, Mazzotta C, Bernardini L, Filippi T, Battaglia A, Donti E, Earl D, Prontera P. Five children with deletions of 1p34. 3 encompassing AGO1 and AGO3 Eur J Hum Genet. 2015; 23(6):761-5. Epub 2014/10/02. doi: 10.1038/ejhg.2014.202. PubMed PMID: 25271087; PMCID: PMC4795073.
23. Okamura K, Ishizuka A, Siomi H, Siomi M C. Distinct roles for Argonaute proteins in small RNA-directed RNA cleavage pathways. Genes Dev. 2004; 18(14):1655-66. Epub 2004/07/03. doi: 10.1101/gad12102204. PubMed PMID: 15231716; PMCID: PMC478188.
24. Wilkins C, Dishongh R, Moore S C, Whitt M A, Chow M, Machaca K. RNA interference is an antiviral defence mechanism in Caenorhabditis elegans. Nature. 2005; 436(7053):1044-7. Epub 2005/08/19. doi: 10.1038/nature03957. PubMed PMID: 16107852.
25. Li H, Li W X, Ding S W. Induction and suppression of RNA silencing by an animal virus. Science. 2002; 296(5571):1319-21. Epub 2002/05/23. doi: 10.1126/science.1070948. PubMed PMID: 12016316.
26. Szittya G, Burgyan J. RNA interference-mediated intrinsic antiviral immunity in plants. Curr Top Microbiol Immunol. 2013; 371:153-81. Epub 2013/05/21. doi: 10.1007/978-3-642-37765-5_6. PubMed PMID: 23686235.
27. Cullen B R, Cherry S, tenOever B R. Is RNA interference a physiologically relevant innate antiviral immune response in mammals? Cell Host Microbe. 2013; 14(4):374-8. Epub 2013/10/22. doi: 10.1016/j.chom.2013.09.011. PubMed PMID: 24139396.
28. Espert L, Degols C, Gongora C, Blondel D, Williams B R, Silverman R H, Mechti N. ISG20, a new interferon-induced RNase specific for single-stranded RNA, defines an alternative antiviral pathway against RNA genomic viruses. J Biol Chem. 2003; 278(18):16151-8. Epub 2003/02/21. doi: 10.1074/jbc.M209628200. PubMed PMID: 12594219.
29. Gongora C, David G, Pintard L, Tissot C, Hua T D, Dejean A, Mechi N. Molecular cloning of a new interferon-induced PML nuclear body-associated protein. J Biol Chem. 1997; 272(31):19457-63. Epub 1997/08/01. doi: 10.1074/jbc.272.3119457. PubMed PMID: 9235947.
30. Stankiewicz A M, Goscik J, Majewska A, Swiergiel A H, Juszczak G R. The Effect of Acute and Chronic Social Stress on the Hippocampal Transcriptome in Mice. PLoS One. 2015; 10(11):e0142195. Epub 2015/11/12. doi: 10.1371/journal.pone.0142195. PubMed PMID: 26556046; PMCID: PMC4640871.
31 Nguyen L H, Espert L, Mechti N, Wilson D M, 3rd. The human interferon- and estrogen-regulated ISG20/HEM45 gene product degrades single-stranded RNA and DNA in vitro. Biochemistry. 2001; 40(24):7174-9. Epub 2001/06/13. PubMed PMID: 11401564.
32. Hauptmann J, Dueck A, Harlander S, Pfaff J, Merkl R, Meister G. Turning catalytically inactive human Argonaute proteins into active slicer enzymes. Nat Struct Mol Biol. 2013; 20(7):814-7. Epub 2013/05/12. doi: 10.1038/nsmb.2577. PubMed PMID: 23665583.
33. Schürmann N, Trabuco L G, Bender C, Russell R B, Grimm D. Molecular dissection of human Argonaute proteins by DNA shuffling. Nat Struct Mol Biol. 2013; 20(7):818-26. Epub 2013/06/09. doi: 10.1038/nsmb.2607. PubMed PMID: 23748378.
34. Nakanishi K, Ascano M, Gogakos T, Ishibe-Murakami S, Serganov A A, Briskin D, Morozov P, Tuschl T, Patel D J. Eukaryote-specific insertion elements control human ARGONAUTE slicer activity. Cell Rep. 2013; 3(6):1893-900. Epub 2013/07/03. doi: 10.1016/j.celrep.2013.06.010. PubMed PMID: 23809764; PMCID: PMC3757560.
35. Schirle N T, MacRae I J. The crystal structure of human Argonaute2. Science. 2012; 336(6084):1037-40. Epub 2012/04/26. doi: 10.1126/science 1221551. PubMed PMID: 22539551: PMCID: PMC3521581.
36. Elkayam E, Kuhn C D, Tocilj A, Haase A D, Greene E M, Hannon G J, Joshua-Tor L. The structure of human argonaute-2 in complex with miR-20a. Cell. 2012; 150(l):100-10. Epub 2012/06/07. doi: 10.1016/j.cell.2012.05.017. PubMed PMID: 22682761; PMCID: PMC3464090.
37. Park M S, Araya-Secchi R, Brackbill J A, Phan H D, Kehling A C, Abd El-Wahab E W, Dayeh D M, Sotomayor M, Nakanishi K. Multidomain Convergence of Argonaute during RISC Assembly Correlates with the Formation of Internal Water Clusters, Mol Cell 2019. Epub 2019/07/22. doi: 10.1016/j.molcel.2019.06.011. PubMed PMID: 31324450.
38. Faehnle C R, Elkayam E, Haase A D, Hannon G J, Joshua-Tor L. The making of a slicer: activation of human Argonaute-1. Cell Rep. 2013; 3(6):1901-9. Epub 2013/06/06. doi: 10.1016/j.celrep.2013.05.033. PubMed PMID: 23746446; PMCID: PMC3769929.
39. Chen J, Liu L, You C, Gu J, Ruan W, Zhang L, Gan J, Cao C, Huang Y, Chen X, Ma J. Structural and biochemical insights into small RNA 3′ end trimming by Arabidopsis SDN1. Nat Commun. 2018; 9(1):3585. Epub 2018/09/06. doi: 10.1038/s41467-018-05942-7. PubMed PMID: 30181559; PMCID: PMC6123492.
40. Yu Y, Ji L, Le B H, Zhai J, Chen J, Luscher E, Gao L, Liu C, Cao X, Mo B, Ma J, Meyers B C, Chen X. ARCONAUTE10 promotes the degradation of miR165/6 through the SDN1 and SDN2 exonucleases in Arabidopsis. PLoS Biol. 2017; 15(2):e2001272. Epub 2017/02/24. doi: 10.1371/journal.pbio.2001272. PubMed PMID: 28231321; PMCID: PMC5322904.
41. Shukla S, Bjerke G A, Muhlrad D, Yi R, Parker R. The RNase PARN Controls the Levels of Specific miRNAs that Contribute to p53 Regulation. Mol Cell. 2019; 73(6):1204-16 e4. Epub 2019/02/17. doi: 10.1016/j.molcel.2019.01.010. PubMed PMID: 30770239; PMCID: PMC6430647.
42. Espert L, Rey C, Gonzalez L, Degols C, Chelbi-Alix M K, Mechti N, Giongora C. The exonuclease ISG20 is directly induced by synthetic dsRNA via NF-kappaB and IRF1 activation. Oncogene. 2004; 23(26):4636-40. Epub 2004/04/06. doi: 10.1038/si.onc.1207586. PubMed PMID: 15064705.
43. Sasaki T, Shiohama A, Minoshima S, Shimizu N. Identification of eight members of the Argonaute family in the human genome. Genomics. 2003; 82(3):323-30. PubMed PMID: 12906857.
44. Schirle N T, Sheu-Gruttadauria J, MacRae I J. Structural basis for microRNA targeting. Science. 2014; 346(6209):608-13. doi: 10.1126/science.1258040. PubMed PMID: 25359968; PMCID: PMC4313529.
45. Flores-Jasso C F, Salomon W E, Zamore P D. Rapid and specific purification of Argonaute-small RNA complexes from crude cell lysates. RNA. 2013; 19(2):271-9. Epub 2012/12/18. doi: 10.1261/rna.036921.112. PubMed PMID: 23249751; PMCID: PMC3543083.
46. Dayeh D M, Cantara W A, Kitzrow J P, Musier-Forsyth K, Nakanishi K. Argonaute-based programmable RNase as a tool for cleavage of highly-structured RNA. Nucleic Acids Res. 2018; 46(16):e98. Epub 2018/06/14. doi: 10.1093/nar/gky496. PubMed PMID: 29897478; PMCID: PMC6144825.
47. Wenzel S, Imasaki T, Takagi Y. A practical method for efficient and optimal production of Seleno-methionine-labeled recombinant protein complexes in the insect cells. Protein Sci. 2019; 28(4):808-22. Epub 2019/01/22. doi: 10.1002/pro.3575. PubMed PMID: 30663186; PMCID: PMC6423997.
48. Llave C, Xie Z, Kasschau K D, Carrington J C. Cleavage of Scarecrow-like mRNA targets directed by a class of Arabidopsis miRNA. Science. 2002; 297(5589):2053-6. Epub 2002/09/21. doi: 10.1126/science.1076311. PubMed PMID: 12242443.
49. Park J H, Shin C. MicroRNA-directed cleavage of targets: mechanism and experimental approaches. BMB Rep. 2014; 47(8):417-23. Epub 2014/05/27. doi: 10.5483/bmbrep.2014.47.8.109. PubMed PMID: 24856832; PMCID: PMC4206712.
50. Kumar P, Anaya J, Mudunuri S B, Dutta A. Meta-analysis of tRNA derived RNA fragments reveals that they are evolutionarily conserved and associate with AGO proteins to recognize specific RNA targets. BMC Biol. 2014; 12:78. Epub 2014/10/02. doi: 10.1186/s12915-014-0078-0. PubMed PMID: 25270025; PMCID: PMC4203973.
51. Sarshad A A, Juan A H, Muler A I C, Anastasakis D G, Wang X, Genzor P, Feng X, Tsai P F, Sun H W, Haase A D, Sartorelli V, Hafner M. Argonaute-miRNA Complexes Silence Target mRNAs in the Nucleus of Mammalian Stem Cells. Mol Cell. 2018; 71(6):1040-50 e8. Epub 2018/08/28. doi: 10.1016/j.molcel.2018.07.020. PubMed PMID: 30146314.
52. Imaizumi T, Mechti N, Matsumiya T, Sakaki H, Kubota K, Yoshida H, Kimura H, Satoh K. Expression of interferon-stimulated gene 20 in vascular endothelial cells. Microbiol Immunol. 2008; 52(1):30-5. Epub 2008/03/21 doi: 10.1111/j.1348-0421.2008.00004.x. PubMed PMID: 18352910.
53. Weiss C M, Trobaugh D W, Sun C, Lucas T M, Diamond M S, Ryman K D, Klimstra W B. The Interferon-Induced Exonuclease ISG20 Exerts Antiviral Activity through Upregulation of Type I Interferon Response Proteins. mSphere. 2018; 3(5). Epub 2018/09/21. doi: 10.1128/mSphere.00209-18. PubMed PMID: 30232164; PMCID: 50 PMC6147134.
54. Shrivastava S, Petrone J, Steele R, Lauer G M, Di Bisceglie A M, Ray R B. Up-regulation of circulating miR-20a is correlated with hepatitis C virus-mediated liver disease progression. Hepatology. 2013; 58(3):863-71 Epub 2013/02/08. doi: 10.1002/hep.26296. PubMed PMID: 23390075; PMCID: PMC3664107.
55. Jiang D, Guo H, Xu C, Chang J, Gu B, Wang L, Block T M, Guo J T. Identification of three interferon-inducible cellular enzymes that inhibit the replication of hepatitis C virus. J Virol. 2008; 82(4):1665-78. Epub 2007/12/14. doi: 10.1128/JVI.02113-07. Pub Med PMID: 18077728; PMCID: PMC2258705.
56. D. P. Bartel, Metazoan MicroRNAs. Cell 173, 20-51 (2018).
57. D. Lee, D. Park, J. H. Park, J. H. Kim, C. Shin, Poly(A)-specific ribonuclease sculpts the 3′ ends of microRNAs. RNA 25, 388-405 (2019).
58. M. Yoda et al., Poly(A)-specific ribonuclease mediates 3-end trimming of Argonaute2-cleaved precursor microRNAs. Cell Rep 5, 715-726 (2013).
59. J. Ross, G. Kobs, G. Brewer, S. W. Peltz, Properties of the exonuclease activity that degrades H4 histone mRNA. J Biol Chem 262, 9374-9381 (1987).
60. Y. Liu et al., Interferon-inducible ribonuclease ISG20 inhibits hepatitis B virus replication through directly binding to the epsilon stem-loop structure of viral RNA. PLoS Pathog 13, e1006296 (2017).
61 S. Iwasaki, Y. Tomari, Reconstitution of RNA Interference Machinery. Methods Mol Biol 1680, 131-143 (2018).
62. W. F. Lima et al., Single-stranded siRNAs activate RNAi in animals. Cell 150, 883-894 (2012).

1. A method of regulating a target nucleic acid using an Argonaute-3 (AGO3) molecule, wherein the AGO3 functions as a slicer of the target nucleic acid, the method comprising:
(a) preparing or isolating a double-stranded RNA molecule, wherein one of the strands comprises sufficient complementarity to hybridize with the target mRNA, wherein said double stranded RNA molecule comprises a cleavage-inducing tyRNA (cityRNA) of 12-16 nucleotides in length;
(b) exposing the double-stranded RNA molecule to an RNA induced silencing complex (RISC) comprising AGO3 under conditions which allow for loading of the double-stranded RNA molecule into RISC;
(c), exposing the AGO3 associated RISC loaded with cityRNA to the target nucleic acid, thereby allowing AGO3-associated RISC to modify the target nucleic acid.

2. The method of claim 1, wherein the target nucleic acid is RNA or DNA.

3. The method of claim 2, wherein the RNA is mRNA.

4. The method of claim 1, wherein the double-stranded RNA is cleaved by Dicer before exposure to RISC.

5. The method of claim 1, wherein the cityRNA has at least 50% complementarity to the target nucleic acid over the length of the strand.

6. The method of claim 1, wherein the cityRNA has at least 70% complementarity to the target nucleic acid over the length of the strand.

7. The method of claim 1, wherein the cityRNA has at least 90% complementarity to the target nucleic acid over the length of the strand.

8. The method of claim 1, wherein the target nucleic acid is cleaved by AGO3-associated RISC loaded with cityRNA.

9. The method of claim 1, wherein the target nucleic acid is silenced by AGO3-associated RISC loaded with cityRNA.

10. The method of claim 9, wherein silencing comprises gene-specific silencing.

11. The method of claim 10, wherein gene-specific silencing comprises transcriptional gene silencing (TGS) activity or a post-transcriptional gene silencing (PTGS) activity.

12. The method of claim 11, wherein said PTGS activity comprises RNA interference and/or translational attenuation.

13. The method of claim 1, wherein modifying the target nucleic acid is used to treat a disease or disorder.

14. The method of claim 13, wherein said disease or disorder is an infectious agent, a cancer, or a genetic defect.

15. The method of claim 1, wherein the double stranded RNA molecule which selectively interacts with AGO3 is prepared in vitro.

16. The method of claim 15, wherein the double stranded RNA molecule is chemically and/or enzymatically synthesized.

17. The method of claim 1, wherein the double stranded RNA molecule which selectively interacts with AGO3 is naturally occurring and isolated prior to exposure to RISC.

18. The method of claim 1, wherein the city RNA is a siRNA, shRNA or a miRNA.

19. The method of claim 1, wherein the cityRNA molecule is 14 nucleotides in length.

20. The method of claim 1, wherein binding of AGO3 to the target nucleic acid is at least 10- to 300 times faster than binding of AGO2.

21. The method of claim 1, wherein the conditions which allow for loading of the double-stranded nucleic acid molecule into RISC include the degradation of one of the double-stranded RNA molecules that is not complementary to the target nucleic acid, thereby forming the single-stranded cityRNA.

22. A single- or double-stranded non-naturally occurring cleavage-inducing tyRNA (cityRNA) of 12-16 nucleotides in length, wherein the cityRNA is capable of activating slicing of AGO3.

23. The non-naturally occurring cityRNA of claim 22, wherein the cityRNA is 14 nucleotides in length.

24-47. (canceled)


SEQUENCE LISTING (86 sequences)

SEQ ID NO: 1 - RNA, 8 nt, Artificial Sequence
  Synthetic Construct
uaaagugc

SEQ ID NO: 2 - RNA, 10 nt, Artificial Sequence
  Synthetic Construct
uaaagugcuu

SEQ ID NO: 3 - RNA, 12 nt, Artificial Sequence
  Synthetic Construct
uaaagugcuuau

SEQ ID NO: 4 - RNA, 13 nt, Artificial Sequence
  Synthetic Construct
uaaagugcuuaua

SEQ ID NO: 5 - RNA, 14 nt, Artificial Sequence
  Synthetic Construct
uaaagugcuuauag

SEQ ID NO: 6 - RNA, 15 nt, Artificial Sequence
  Synthetic Construct
uaaagugcuuauagu

SEQ ID NO: 7 - RNA, 16 nt, Artificial Sequence
  Synthetic Construct
uaaagugcuuauagug

SEQ ID NO: 8 - RNA, 23 nt, Artificial Sequence
  Synthetic Construct
uaaagugcuuauagugcagguag

SEQ ID NO: 9 - RNA, 33 nt, Artificial Sequence
  Synthetic Construct
aucuaccauuucacgaauaucacguccauccag

SEQ ID NO: 10 - RNA, 14 nt, Artificial Sequence
  Synthetic Construct
uaaagugcuuauag

SEQ ID NO: 11 - RNA, 23 nt, Artificial Sequence
  Synthetic Construct
uaaagugcuuauagugcagguag

SEQ ID NO: 12 - RNA, 23 nt, Artificial Sequence
  Synthetic Construct
uaaagugcuuauagugcagguag

SEQ ID NO: 13 - RNA, 23 nt, Artificial Sequence
  Synthetic Construct
accugcacuauaagcacuuuaag

SEQ ID NO: 14 - RNA, 14 nt, Artificial Sequence
  Synthetic Construct
uaaagugcuuauag

SEQ ID NO: 15 - RNA, 23 nt, Artificial Sequence
  Synthetic Construct
uaaagugcuuauagugcagguag

SEQ ID NO: 16 - RNA, 23 nt, Artificial Sequence
  Synthetic Construct
uaaagugcuuauagugcagguag

SEQ ID NO: 17 - RNA, 23 nt, Artificial Sequence
  Synthetic Construct
accugcacuauaagcacuuuaag

SEQ ID NO: 18 - RNA, 14 nt, Artificial Sequence
  Synthetic Construct
uaaagugcuuauag

SEQ ID NO: 19 - RNA, 14 nt, Artificial Sequence
  Synthetic Construct
ugagguaguagguu

SEQ ID NO: 20 - RNA, 21 nt, Artificial Sequence
  Synthetic Construct
ugagguaguagguuguauagu

SEQ ID NO: 21 - RNA, 31 nt, Artificial Sequence
  Synthetic Construct
aucuaccacuccaucauccaacauaucacag

SEQ ID NO: 22 - RNA, 14 nt, Artificial Sequence
  Synthetic Construct
uagcagcacguaaa

SEQ ID NO: 23 - RNA, 22 nt, Artificial Sequence
  Synthetic Construct
uagcagcacguaaauauuggcg

SEQ ID NO: 24 - RNA, 32 nt, Artificial Sequence
  Synthetic Construct
aucuaccaucgucgugcauuuauaaccgccag

SEQ ID NO: 25 - RNA, 14 nt, Artificial Sequence
  Synthetic Construct
ugugcaaauccaug

SEQ ID NO: 26 - RNA, 23 nt, Artificial Sequence
  Synthetic Construct
ugugcaaauccaugcaaaacuga

SEQ ID NO: 27 - RNA, 33 nt, Artificial Sequence
  Synthetic Construct
aucuaccacacguuuagguacguuuugacucag

SEQ ID NO: 28 - RNA, 14 nt, Artificial Sequence
  Synthetic Construct
caaagugcuuacag

SEQ ID NO: 29 - RNA, 23 nt, Artificial Sequence
  Synthetic Construct
caaagugcuuacagugcagguag

SEQ ID NO: 30 - RNA, 33 nt, Artificial Sequence
  Synthetic Construct
aucuaccauuucacgaaugucacguccauccag

SEQ ID NO: 31 - RNA, 14 nt, Artificial Sequence
  Synthetic Construct
uaaggugcaucuag

SEQ ID NO: 32 - RNA, 23 nt, Artificial Sequence
  Synthetic Construct
uaaggugcaucuagugcagauag

SEQ ID NO: 33 - RNA, 33 nt, Artificial Sequence
  Synthetic Construct
aucuaccauuccacguagaucacgucuauccag

SEQ ID NO: 34 - RNA, 14 nt, Artificial Sequence
  Synthetic Construct
ugugcaaaucuaug

SEQ ID NO: 35 - RNA, 23 nt, Artificial Sequence
  Synthetic Construct
ugugcaaaucuaugcaaaacuga

SEQ ID NO: 36 - RNA, 33 nt, Artificial Sequence
  Synthetic Construct
aucuaccacacguuuagauacguuuugacucag

SEQ ID NO: 37 - RNA, 14 nt, Artificial Sequence
  Synthetic Construct
uucacaguggcuaa

SEQ ID NO: 38 - RNA, 21 nt, Artificial Sequence
  Synthetic Construct
uucacaguggcuaaguuccgc

SEQ ID NO: 39 - RNA, 31 nt, Artificial Sequence
  Synthetic Construct
aucuaccaagugucaccgauucaaggcgcag

SEQ ID NO: 40 - RNA, 14 nt, Artificial Sequence
  Synthetic Construct
uauugcacuugucc

SEQ ID NO: 41 - RNA, 22 nt, Artificial Sequence
  Synthetic Construct
uauugcacuugucccggccugu

SEQ ID NO: 42 - RNA, 32 nt, Artificial Sequence
  Synthetic Construct
aucuaccauaacgugaacagggccggacacag

SEQ ID NO: 43 - DNA, 37 bp, Artificial Sequence
  Synthetic Construct
aucuaccauuucacgaauaucacguccauccataagg

SEQ ID NO: 44 - RNA, 23 nt, Artificial Sequence
  Synthetic Construct
uaaagugcuuauagugcagguag

SEQ ID NO: 45 - RNA, 31 nt, Artificial Sequence
  Synthetic Construct
aucuaccauuucacgaauaucacguccaucg

SEQ ID NO: 46 - RNA, 23 nt, Artificial Sequence
  Synthetic Construct
uaaagugcuuauagugcagguag

SEQ ID NO: 47 - DNA, 30 bp, Artificial Sequence
  Synthetic Construct
auuucacgaauaucacguccauccataagg

SEQ ID NO: 48 - RNA, 23 nt, Artificial Sequence
  Synthetic Construct
uaaagugcuuauagugcagguag

SEQ ID NO: 49 - RNA, 14 nt, Artificial Sequence
  Synthetic Construct
uaaagugcuuauag

SEQ ID NO: 50 - RNA, 14 nt, Artificial Sequence
  Synthetic Construct
ugagguaguagguu

SEQ ID NO: 51 - RNA, 14 nt, Artificial Sequence
  Synthetic Construct
uagcagcacguaaa

SEQ ID NO: 52 - RNA, 14 nt, Artificial Sequence
  Synthetic Construct
ugugcaaauccaug

SEQ ID NO: 53 - RNA, 14 nt, Artificial Sequence
  Synthetic Construct
uaaagugcuuauag

SEQ ID NO: 54 - RNA, 14 nt, Artificial Sequence
  Synthetic Construct
uaaagugcuuauag

SEQ ID NO: 55 - RNA, 19 nt, Artificial Sequence
  Synthetic Construct
acauuucacgaauaucacg

SEQ ID NO: 56 - RNA, 14 nt, Artificial Sequence
  Synthetic Construct
uacagugcuuauag

SEQ ID NO: 57 - RNA, 19 nt, Artificial Sequence
  Synthetic Construct
acaugucacgaauaucacg

SEQ ID NO: 58 - RNA, 14 nt, Artificial Sequence
  Synthetic Construct
uagcagcacguaaa

SEQ ID NO: 59 - RNA, 14 nt, Artificial Sequence
  Synthetic Construct
uagcagcacguaaa

SEQ ID NO: 60 - RNA, 14 nt, Artificial Sequence
  Synthetic Construct
uagcagcacguaaa

SEQ ID NO: 61 - RNA, 14 nt, Artificial Sequence
  Synthetic Construct
uaaagugcuuauac

SEQ ID NO: 62 - RNA, 14 nt, Artificial Sequence
  Synthetic Construct
uaaagugcuuauug

SEQ ID NO: 63 - RNA, 14 nt, Artificial Sequence
  Synthetic Construct
uaaagugcuuaaag

SEQ ID NO: 64 - RNA, 14 nt, Artificial Sequence
  Synthetic Construct
uaaagugcuuuuag

SEQ ID NO: 65 - RNA, 14 nt, Artificial Sequence
  Synthetic Construct
uaaagugcuaauag

SEQ ID NO: 66 - RNA, 14 nt, Artificial Sequence
  Synthetic Construct
uaaagugcauauag

SEQ ID NO: 67 - RNA, 14 nt, Artificial Sequence
  Synthetic Construct
uaaagugguuauag

SEQ ID NO: 68 - RNA, 14 nt, Artificial Sequence
  Synthetic Construct
uaaaguccuuauag

SEQ ID NO: 69 - RNA, 14 nt, Artificial Sequence
  Synthetic Construct
uaaagagcuuauag

SEQ ID NO: 70 - RNA, 14 nt, Artificial Sequence
  Synthetic Construct
uaaacugcuuauag

SEQ ID NO: 71 - RNA, 14 nt, Artificial Sequence
  Synthetic Construct
uaaugugcuuauag

SEQ ID NO: 72 - RNA, 14 nt, Artificial Sequence
  Synthetic Construct
uauagugcuuauag

SEQ ID NO: 73 - RNA, 14 nt, Artificial Sequence
  Synthetic Construct
uaaagugcuuauag

SEQ ID NO: 74 - RNA, 14 nt, Artificial Sequence
  Synthetic Construct
auaagugcuuauag

SEQ ID NO: 75 - RNA, 14 nt, Artificial Sequence
  Synthetic Construct
uaaagugcuuauag

SEQ ID NO: 76 - RNA, 24 nt, Artificial Sequence
  Synthetic Construct
aucuaccauuucacgaauaucacg

SEQ ID NO: 77 - DNA, 35 bp, Artificial Sequence
  Synthetic Construct
aucuaccauuucacgaauaucacguccauccaaag

SEQ ID NO: 78 - RNA, 14 nt, Artificial Sequence
  Synthetic Construct
uaaagugcuuauag

SEQ ID NO: 79 - RNA, 31 nt, Artificial Sequence; identical to SEQ ID NO: 45
  Synthetic Construct

SEQ ID NO: 80 - RNA, 14 nt, Artificial Sequence
  Synthetic Construct
uaaagugcuuauag

SEQ ID NO: 81 - DNA, 30 bp, Artificial Sequence; identical to SEQ ID NO: 47
  Synthetic Construct

SEQ ID NO: 82 - RNA, 14 nt, Artificial Sequence
  Synthetic Construct
uaaagugcuuauag

SEQ ID NO: 83 - RNA, 24 nt, Artificial Sequence
  Synthetic Construct
auuucacgaauaucacguccaucg

SEQ ID NO: 84 - RNA, 14 nt, Artificial Sequence
  Synthetic Construct
uaaagugcuuauag

SEQ ID NO: 85 - RNA, 15 nt, Artificial Sequence
  Synthetic Construct
auuucacgaauaucg

SEQ ID NO: 86 - RNA, 14 nt, Artificial Sequence
  Synthetic Construct
uaaagugcuuauag